SEQUENCE LISTING (4 sequences)

SEQ ID NO: 1 - protein, 6 aa, Artificial
  Synthetic peptide
VQAALD

SEQ ID NO: 2 - protein, 6 aa, Artificial
  Synthetic peptide
VQEALD

SEQ ID NO: 3 - protein, 45 aa, Artificial
  Synthetic peptide
VRNADVIAGFDDGVIVEKGNHDELMKEKGIYFKLVTMQTAGNEVE

SEQ ID NO: 4 - protein, 36 aa, Artificial
  Synthetic peptide
CFITKALGISYGRKKRRQRRRPPQGSQTHQVSLSKQ

(19) United States
(12) Patent Application Publication
(10) Pub. No.: US 2010/0196403 A1
(43) Pub. Date: Aug. 5, 2010

(54) ANTIBODY CONJUGATES FOR CIRCUMVENTING MULTI-DRUG RESISTANCE

(76) Inventors: Jacob Hochman, Jerusalem (IL); Hans Wellhoener, Hannover (DE)

Correspondence Address: WINSTON & STRAWN LLP;PATENT DEPARTMENT, 1700 K STREET, N.W., WASHINGTON, DC, 20006, US

(21) Appl. No.: 12/523,767

(22) PCT Filed: Jan. 27, 2008
(86) PCT No.: PCT/IL08/00117

§ 371 Date: Apr. 12, 2010

Related U.S. Application Data
(60) Provisional application No. 60/897,871, filed on Jan. 29, 2007.

(51) Int. Cl. A61K 39/395; C07K 16/00; C12N 5/00; A61P 35/00

(52) U.S. Cl. 424/178.1; 530/391.1; 435/375

(57) ABSTRACT

Conjugates of a cell permeability moiety coupled to an antibody against an intracellular epitope of a multi drug resistance (MDR) protein are provided. Also provided are pharmaceutical compositions that include these conjugates and methods for their use in preventing and inhibiting multi drug resistance to therapeutic agents, particularly to chemotherapeutic agents.

### FIELD OF THE INVENTION

The present invention is in the field of cancer therapy and in particular inhibition of multi-drug resistance (MDR) using cell permeable conjugates of antibodies against intracellular epitopes of MDR proteins.

### BACKGROUND

### Multi-Drug Resistance

Tumor cells become resistant against chemotherapy after prolonged treatment. The resistance may be intrinsic or acquired resistance and is known to be a major contributing factor to failure in cancer treatment. Clinical drug resistance often presents as a multi-drug resistance (MDR) phenotype, characterized as de novo resistance to a variety of structurally diverse drugs or as developed cross-resistance to chemotherapeutic agents that have never been used in previous chemotherapy

Although the cellular basis underlying drug resistance is not fully understood, several factors have been identified that contribute to its development. These include drug efflux mechanisms, increased drug inactivation (e.g. glutathione-S-transferase and resistance to allylating agents), drug target mutation (topoisomerase mutation), altered DNA repair and resistance to apoptosis (p53 mutation, bcl-2 over expression etc.).

As will be appreciated, MDR is largely due to protein molecules residing in the cell membrane (cytoplasmic membrane), the so-called Multi-Drug Resistant proteins (MDR proteins). They meander back and forth through the cell membrane, forming loops on its extracellular as well as on its intracellular side. They “pump out” cytostatic (and other lipophilic) molecules once such molecules have entered a cell. Tumor cells defend themselves against the prolonged attack of cytostatic drugs by producing more MDR protein molecules.

Clinical drug resistance may be caused by any one or a combination of the above mechanisms. Increased transmembrane efflux of xenobiotics is one of the best characterized mechanisms of MDR and is known to be mediated through over-expression of adenosine triphosphate ATP-binding cassette (ABC) transporter superfamily members such as P-glycoprotein (P-gp MDR1 ABCB1), multidrug resistance associated protein (MRP1), or breast cancer 1 resistance protein (BCRP).

P-gp is the most extensively studied of these transporters. P-gp is encoded by the mdr1 gene and found to be over-expressed in many tumor cells, including a variety of leukemias and solid tumors. P-gp over-expression provides protection against a number of chemotherapeutic agents including anthracyclines, vinca alkaloids, anthracenes derivatives, epipodophyllotoxins, and tubulin polymerizing drugs. More specifically, P-gp is an ATP-powered outward pump of lipophilic substrates (Fardel et al 1996, Gottesman et al 2002). The 170 kD glycoprotein consists of four hydrophobic domains arranged in the sequence NH₂-TMD1-NBD1-linker peptide-TMD2-NBD2-COOH (NBD denoted nuclear binding domain). Each transmembrane domain TMD has six membrane segments and binds neutral or positively charged lipophilic substrates. Each nuclear binding domain NBD extends into the cytoplasm and presents one cytoplasmic ATP binding site. When a substrate binds to a TMD, the ensuing conformational change is transmitted to the NBD. In response, the ATP turnover at the NBD increases and the energy liberated by ATP-hydrolysis is fed back to the TMD. This leads to an increased outward transport of the bound substrate.

Numerous attempts have been made to inhibit P-gp with appropriate substances. Well known examples are verapamil (used to treat cardiovascular diseases) and cyclosporin (used in organ transplantation). Either substance inhibits P-gp effectively only at concentrations that would evoke severe side effects in patients. In a search for substances with much lower side effects, monoclonal antibodies (MABs) directed against P-gp have been produced. So far, the MABs tested in animals and patients were raised against epitopes on the extracellular loops of MDR proteins. Positive results from animal experiments and treated patients were reported. However, there was no clinical follow-up. The epitopes on the extracellular loops may be less important for the function of the MDR proteins.

Inhibitors of the MDR activity may be arranged in three groups. To the first group belong low-molecular weight substances (Stein, W. D. 2002). A few of them are now under clinical investigation (Robert, J., and Jarry, C. 2003, Gottesman, M. M. 2002). The second group comprises peptides and antibody fragments acting from the outside of the cells. Recent examples are peptide transmembrane inhibitors and recombinant single-chain Fv antibody fragments (Haus-Cohen, et al., 2004, Tarasova et al., 2005). The third group comprises substances acting from the intracellular space: antisense oligonucleotides (Motomura et al., 1998, Alahari et al., 1998, Cucco et al., 1996), ribozymes (Materna et al., 2005, Osada et al., 2003, Scanlon et al., 1994), C219 sFvs expression vectors (Heike et al., 2001), and antibodies (Mie et al., 2003).

It was reported (Heike et al., 2001), that transfection of a vector coding for single-chain Fv from the C219 monoclonal antibody with lipofectamin followed by intracellular expression of this construct, overcame multi-drug resistance, thereby increasing adriamycin uptake and rhodamine retention.

### Tat Conjugates

Tat protein directs key events of the HIV-1 life cycle (Sodroski et al. 1986, Terwillinger et al. 1988). Tat is a small karyophilic protein, which function in the nuclei of infected cells by binding to specific viral RNA elements via a similar peptide motif. It has been demonstrated that linear peptides bearing the Tat Arginine rich motif (Suzuki et al. 2002) are able to penetrate membranes of cultured cells and to accumulate within their nuclei. It was further shown that a conjugate between anti-tetanus antibodies and the fragment 37-72 of HIV-1 Tat protein was taken up by chromaffin cells and that thereafter the translocated antibodies neutralize tetanus toxin inside the cells (Stein et al. 1999). Both the antigen (tetanus toxin) and the antibodies were soluble within the cell, i.e. the antigen as well as the antibody molecules could freely move and interact. An indirect method for the intracellular delivery of antibodies using a Tat-staphylococcal protein-A loaded with fluorescent IgG has also been described (Mie et al., 2003).

U.S. Pat. No. 5,369,009 describes antibody against external epitope of P-gp and uses thereof. The disclosed antibody does not substantially increase the intracellular accumulation or the cytoxicity of either daunomycin or vinblastine in multidrug resistant cells.

WO 96/04313 discloses polyspecific immunoconjugates and antibody composites for targeting the multidrug resistant phenotype. The conjugates disclosed comprise: at least one antibody component directed against MDR protein epitope, at least one antibody component directed against a tumor or infectious agent determinant, and at least one diagnostic or therapeutic agent.

WO 93/25700 and WO 93/19094 disclose antibodies against extracellularly-located epitope of human P-gp.

C219, disclosed by Georges et al. in 1990, is a commercially available murine monoclonal antibody directed against the nuclear binding domain (NBD) of P-gp. It binds to the sequence VVQAALD (565-571) in NBD1 and to VVQEALD (1210-1216) in NBD2. WO 90/15330 and its US counterpart U.S. Pat. No. 5,223,400 disclosed immunoassay for determining the specificity of binding of this, among others, antibody to its antigen P-gp. C219 has been shown to inhibit the ATPase activity (and thereby the provision of energy) in membrane preparations of multidrug-resistant cells (Kokubu et al. 1997).

There is an unmet need for providing improved therapeutic agents capable of augmenting intracellular activity of chemotherapeutic compounds by specific inhibition of MDR efflux activity.

### SUMMARY OF THE INVENTION

The present invention is based on the surprising finding that anti MDR antibodies directed against intracellular epitopes, linked to a translocator peptide can enter intact cells and reduce the biological activity of membrane-bound MDR proteins. It was unexpectedly found that conjugation of antibodies directed against intracellular epitopes of MDR proteins, or fragments thereof, with moieties capable of increasing the permeability of molecules into cells, results in inhibition of MDR activity. Without wishing to be bound to any mechanism or theory of action it is hypothesized that the conjugates may act by binding to the intracellular epitope domains of the MDR protein and blocking the activity of an ATP-dependent efflux pump.

It is further provided that conjugates comprising an antibody against an intracellular epitope of an MDR protein or an active fragment thereof, and a cell permeability moiety are capable of temporarily opening the Blood-Brain-Barrier (BBB) by impairing the activity of P-glycoprotein (P-gp), an MDR protein that is normally expressed at high levels in the BBB, thus enhancing the transport of agents, including chemotherapeutic agents, into the brain and subsequently treating the brain for example where the agents are chemotherapeutic agents, impairing growth of brain tumors.

According to one aspect the present invention provides a conjugate comprising (i) an antibody, or a fragment thereof comprising at least the antigen-binding portion, capable of binding an intracellular epitope of an MDR protein within intact cells, and inhibiting said protein activity, (ii) a cell entering moiety; and optionally (iii) a linker connecting (i) and (ii).

According to one embodiment, the MDR protein is an ATP-binding cassette (ABC) transporter selected from the group consisting of: MDR1 (ABCB1, P-gp), MRP4 (ABCC4), MRP5 (ABCC5), MRP1 (ABCC1), MRP2 (ABCC2), MRP3 (ABCC3), and MXR/BCRP/ABC-p (ABCG2). According to a specific embodiment the MDR protein is the protein MDR1 (ABCB1, P-gp).

According to some embodiments the antibody or antibody fragment is directed against a Pg-P epitope comprising a sequence selected from VQAALD (SEQ ID NO:1) and VQEALD (SEQ ID NO:2). According to a specific embodiment the antibody is the C219 antibody.

According to certain embodiments, the intracellular epitope of the MDR protein is within the sequence of residues 596-636 of MDR1 protein having the sequence: VRNADVIAGFDDGVIVEKGNHDELMKEKGIYFKLVTMQTAGNEVE (SEQ ID NO:3).

Any moiety known in the art to actively or passively facilitate or enhance entry of the compound into cells may be used for conjugation with the antibody according to the present invention. Non-limitative examples include: transporter peptides, pore forming peptides, hydrophobic moieties such as fatty acids, steroids and bulky aromatic or aliphatic compounds; moieties which can bind to cell-membrane receptors or carriers, such as steroids, vitamins and sugars, and natural and non-natural amino acids.

Also included within the scope of the present application are compositions comprising an antibody according to the present invention included in a construct capable of facilitating or increasing entry through membranes. Non-limiting examples for such construct include liposomes, encapsulation, nanoparticles, and polymers.

According to one embodiment the cell entering moiety comprises a cationic protein transduction domain (PTD). According to certain embodiments the cell entering moiety comprises an HIV-1 protein-derived peptide. According to a specific embodiment, the cell entering moiety is an HIV-1 Tat-derived peptide. According to a more specific embodiment, the cell entering moiety comprises a peptide comprising amino acids 37-72 of HIV-1 Tat protein having the sequence CFITKALGISYGRKKRRQRRRPPQGSQTHQVSLSKQ (SEQ ID NO: 4), or an analog thereof.

Conjugates according to the present invention may be formed, directly or through a linker, between any functional group of the antibody or active fragment thereof and a functional group of the cell entering moiety. The optional connective linker may be of varied lengths and conformations comprising any suitable chemistry including but not limited to amine, amide, carbamate, thioether, oxyether, sulfonamide bond and the like. Non-limiting examples for such linkers include amino acids, sulfone amide derivatives, amino thiol derivatives and amino alcohol derivatives.

According to a specific embodiment, the cell-permeability moiety is connected to antibody or antibody fragment via a disulfide bond between an SH group of the antibody or antibody fragment and a SH group in the cell entering moiety. According to a specific embodiment the cell-permeability moiety is connected via a disulfide bond to a carbohydrate group of an antibody or a fragment thereof comprising at least the antigen-binding portion.

According to certain embodiments the linker comprises a cleavable sequence. According to one embodiment the cleavable linker is cleaved by intracellular enzymes. According to a specific embodiment the cleavable linker is cleaved by intracellular enzymes over-expressed in cancer cells. According to a more specific embodiment the cleavable linker comprises a protease specific cleavable sequence, wherein the protease is more abundant in malignant cells or secreted by malignant cells more than normal cells.

According to a specific embodiment an antibody directed against an intracellular epitope of the P-gp protein, or a fragment thereof comprising at least the antigen-binding portion, is conjugated to a cell-permeability moiety. According to a specific embodiment the antibody or antibody fragment is connected via a cleavable bond or linker. According to a more specific embodiment the cleavable bond is a disulfide bond. According to yet another specific embodiment the cell permeability moiety is a Tat protein fragment comprising the sequence CFITKALGISYGRKKRRQRRRPPQGSQTHQVSLSKQ (SEQ ID NO: 4) or an analog thereof.

According to one embodiment of the present invention, the antibody is a monoclonal antibody. According to a specific embodiment the monoclonal antibody is selected from the group consisting of: humanized antibody, human antibody, chimeric antibody and an antibody fragment comprising at least the antigen-binding portion of an antibody. According to a specific embodiment the antibody fragment is selected from the group consisting of: Fab, Fab′, F(ab′)₂, Fd, Fd′, Fv, dAb, isolated CDR region, single chain antibody, “diabody”, and “linear antibody”.

In another aspect the present invention provides a pharmaceutical composition useful for preventing, attenuating or treating a disease or disorder associated with MDR resistance. The pharmaceutical composition of the present invention is particularly useful for treating cancer. In particular, the pharmaceutical composition is useful for i) circumventing or treating multi-drug resistant cancer and ii) preventing resistance from developing in cancer cells.

The cancer according to the invention can be a solid tumor or a hematological malignancy. The term cancer includes any cancer including, without limitation, ovarian cancer, pancreatic cancer, head and neck cancer, squamous cell carcinoma, gastrointestinal cancer, breast cancer (such as carcinoma, ductal, lobular, and nipple), prostate cancer, non small cell lung cancer, Non-Hodgkin's lymphoma, multiple myeloma, leukemia (such as acute lymphocytic leukemia, chronic lymphocytic leukemia, acute myelogenous leukemia, and chronic myelogenous leukemia), brain cancer, neuroblastoma, and sarcomas. In a preferred example the cancer involves cell over-expression of P-glycoprotein.

According to a specific embodiment, the invention concerns treating cancer associated with MDR (ABC transporters in human cancers) such as, without being limited thereto, colon, kidney, adrenocortical and hepatocellular cancers; Breast cancer, Acute Myelogenous Leukemia (AML), chronic lymphocitic leukemia (CLL), pro-lymphocitic leukemia, oesophagal carcinoma, non-small-cell lung cancers, soft-tissue sarcomas and osteosarcomas.

According to another embodiment, the pharmaceutical composition according to the present invention is used for delivering medicinal or diagnostic agents into the brain. According to one embodiment the medicinal agent is a chemotherapeutic agent used for inhibition or treatment of brain tumor or brain metastases.

According to yet another embodiment the condition associated with MDR resistance is resistance to drugs used to treat or prevent HIV infection or AIDS.

According to one embodiment the pharmaceutical composition comprises a therapeutically effective amount of a conjugate comprising (i) an antibody, or a fragment thereof comprising at least the antigen-binding portion, capable of binding an intracellular epitope of an MDR protein and inhibiting said protein activity, (ii) a cell entering moiety; and optionally (iii) a linker connecting (i) and (ii).

The pharmaceutical composition according to the present invention may be administered to a subject in need thereof as part of a treatment regimen in conjunction with an anti-neoplastic composition. The pharmaceutical composition according to the present invention may be administered together with the anti-neoplastic composition or separately.

According to a certain embodiments, the pharmaceutical composition according to the present invention may further comprise an anti-cancer agent of the type that is expelled by the specific MDR (ATP-binding cassette transporter) against which the antibody part of the conjugate is directed. The anti-cancer agent may be present in a mixture with the conjugate of the invention or the conjugate and anti-cancer agent may be present in different carriers. According to a specific embodiment the pharmaceutical composition is provided in the form of a kit with two separate dosage forms and vessels one containing the anti-cancer agent and one the conjugate of the invention. The kit may further comprise instructions for the administration of the two components, either simultaneously, or instructions for the anti-cancer drug to be administered after the conjugate of the invention administered (so as to allow the conjugate to inhibit the ATP-binding cassette (ABC) transporter prior to administration of the anti-cancer agent).

The conjugate of the invention may be administered in a convenient manner such as by injection (e.g. subcutaneous, intravenous, intralesional) or any other suitable route.

According to a specific embodiment the anti-neoplastic or anti-cancer composition comprises at least one chemotherapeutic agent. The chemotherapy agent, which could be administered together with the conjugate according to the present invention, may comprise any such agent known in the art exhibiting anticancer activity, including but not limited to: mitoxantrone, topoisomerase inhibitors, spindle poison vincas: vinblastine, vincristine, vinorelbine (taxol), paclitaxel, docetaxel; alkylating agents: mechlorethamine, chlorambucil, cyclophosphamide, melphalan, ifosfamide; methotrexate; 6-mercaptopurine; 5-fluorouracil, cytarabine, gemcitabin; podophyllotoxins: etoposide, irinotecan, topotecan, dacarbazin; antibiotics: doxorubicin (adriamycin), bleomycin, mitomycin; nitrosoureas: carmustine (BCNU), lomustine, epirubicin, idarubicin, daunorubicin; inorganic ions: cisplatin, carboplatin; interferon, asparaginase; hormones: tamoxifen, leuprolide, flutamide, and megestrol acetate.

According to a specific embodiment, the chemotherapeutic agent is selected from the group consisting of alkylating agents, antimetabolites, folic acid analogs, pyrimidine analogs, purine analogs and related inhibitors, vinca alkaloids, epipodopyllotoxins, antibiotics, L-asparaginase, topoisomerase inhibitor, interferons, platinum coordination complexes, anthracenedione substituted urea, methyl hydrazine derivatives, adrenocortical suppressant, adrenocorticosteroides, progestins, estrogens, antiestrogen, androgens, antiandrogen, and gonadotropin-releasing hormone analog. According to another embodiment, the chemotherapeutic agent is selected from the group consisting of 5-fluorouracil (5-FU), leucovorin (LV), irenotecan, oxaliplatin, capecitabine, paclitaxel and doxetaxel. Two or more chemotherapeutic agents can be used in a cocktail to be administered in combination with administration of the conjugate according to the present embodiment. According to one embodiment the combination chemotherapy is fluorouracil-based, comprising 5-FU and one or more other chemotherapeutic agent(s).

In yet another aspect the present invention is related to a method of preventing, attenuating or treating a disease or disorder associated with multi drug resistance, comprising administering to a subject in need thereof a pharmaceutical composition comprising a therapeutically effective amount of a conjugate comprising an antibody, or a fragment thereof comprising at least the antigen-binding portion, capable of binding an intracellular epitope of an MDR protein and inhibiting said protein activity, and a cell entering moiety optionally connected via a linker; and a pharmaceutically acceptable carrier.

According to yet another aspect, the invention provides a method of treating or inhibiting a disease or disorder associated with MDR resistance, in a subject, comprising administering to the subject effective amounts of a conjugate comprising an antibody, or a fragment thereof comprising at least the antigen-binding portion, capable of binding an intracellular epitope of an MDR protein and inhibiting said protein activity, a cell entering moiety; and optionally a linker connecting, together with an anti-neoplastic composition. According to one embodiment the disease or disorder is cancer. According to another embodiment the disorder is a brain disorder and the conjugate is used for delivering agents through the BBB. According to yet another embodiment the disorder is resistance to drugs used to treat or prevent HIV infection or AIDS.

The cancer amendable for treatment by the present invention include, but not limited to, carcinoma, lymphoma, blastoma, sarcoma, and leukemia or lymphoid malignancies. More particular examples of such cancers include squamous cell cancer, lung cancer (including small-cell lung cancer, non-small cell lung cancer, adenocarcinoma of the lung, and squamous carcinoma of the lung), cancer of the peritoneum, hepatocellular cancer, gastric or stomach cancer (including gastrointestinal cancer), pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney or renal cancer, liver cancer, prostate cancer, vulval cancer, thyroid cancer, hepatic carcinoma and various types of head and neck cancer, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macro globulinemia); chronic lymphocytic leukemia (CLL); acute lymphoblastic leukemia (ALL); Hairy cell leukemia; chronic myeloblastic leukemia; and post-transplant lymphoproliferative disorder (PTLD), as well as abnormal vascular proliferation associated with phakomatoses, edema (such as that associated with brain tumors), and Meigs' syndrome. Preferably, the cancer is selected from the group consisting of colon, kidney, adrenocortical and hepatocellular cancers; breast cancer, Acute Myelogenous Leukemia (AML), Chronic lymphocitic leukemia (CLL), pro-lymphocitic leukemia, oesophagal carcinoma, non-small-cell lung cancers, soft-tissue sarcomas and osteosarcomas.

According to yet another aspect, the invention provides a method of inhibiting MDR activity in MDR cells; the method comprises providing MDR cells with an amount of a conjugate according to the invention, the amount being sufficient to inhibit MDR activity in the cells. According to one embodiment, the conjugate is administered to said cells in combination with one or more chemotherapeutic drugs. The term “combination” used herein denotes, administration of the chemotherapeutic drug to said cells, before, together or after administration of said conjugate, the time gap between administrations of the conjugate of the invention and the chemotherapeutic drug being determined so as to achieve reversal, inhibition or prevention of MDR activity in said cells.

The present invention further provides a method for circumventing or treating MDR cancer, the method comprises providing a subject in need an amount of the conjugate of the invention, the amount being effective to inhibit MDR activity in cancer cells in said subject.

The present invention further provides a method sensitizing an MDR cancer to anti-cancer drugs the method comprises administering said of the invention combination with said anti-cancer drug the amount being effective to sensitize the MDR cancer cells to one or more drugs forming part of the anti-cancer therapy. Preferably the drug should be of the type expelled by the specific ABS transporter, to which the antibody of the conjugate of the invention binds.

The present invention also concerns a method of preventing the development of MDR in a subject undergoing anti-cancer therapy, comprising administering to the subject prior or at the time of the anti-cancer therapy, a therapeutically effective amount of the conjugate of the invention.

The present invention provides a method for treating a subject having cancer, comprising administering to the subject effective amounts of a composition comprising a conjugate comprising (i) an antibody, or a fragment thereof comprising at least the antigen-binding portion, capable of binding an intracellular epitope of an MDR protein and inhibiting said protein activity, (ii) a cell entering moiety; and optionally (iii) a linker connecting (i) and (ii), and an anti-neoplastic composition, whereby co-administration of the conjugate and the anti-neoplastic composition effectively increases the response rate in the group of subjects.

In yet another aspect, the present invention provides a method for increasing the duration of response of a subject having cancer, comprising administering to the subject effective amount of a composition comprising a conjugate according to the invention, and an anti-neoplastic composition, wherein said anti-neoplastic composition comprises at least one chemotherapeutic agent, whereby co-administration of the conjugate and the anti-neoplastic composition effectively increases the duration of response.

Another aspect of the present invention relates to the use of a conjugate comprising (i) an antibody, or a fragment thereof comprising at least the antigen-binding portion, capable of binding an intracellular epitope of an MDR protein and inhibiting said protein activity, (ii) a cell entering moiety; and optionally (iii) a linker connecting (i) and (ii), for the manufacture of a therapeutic composition for the treatment of a disease or disorder associated with multi drug resistance.

According to another aspect of the present invention, use of a conjugate comprising (i) an antibody, or a fragment thereof comprising at least the antigen-binding portion, capable of binding an intracellular epitope of an MDR protein and inhibiting said protein activity, (ii) a cell entering moiety; and optionally (iii) a linker connecting (i) and (ii), for treatment of a disorder or disease associated with MDR resistance, is disclosed. According to one embodiment, the disease or disorder is cancer. According to another embodiment the disease or disorder is resistance to drugs used to treat or prevent HIV infection or AIDS.

The invention further provides a method of enhancing transport of a medicinal agent through the blood brain barrier; the method comprises administering to an individual in need of such treatment a therapeutically effective amount of the conjugate of the invention, in combination with one or more medicinal drugs. According to a preferred embodiment the medicinal agent is a chemotherapeutic agent used to treat brain tumors. The term “in combination” may refer to simultaneous administration of the conjugate and the medicinal agent, or to sequential administration of the two. The order of administration and the time lag between the two administrations should be calculated by bringing into consideration pharmacokinetic considerations.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### DESCRIPTION OF FIGURES

FIG. 1 contains images representing time course of uptake of (37-72)Tat-S-IgGₕᵤₘ-Alexa568 by Ch(R)B30 cells incubated in 1 μM of the thioether conjugate. A—immediately after addition of the conjugate, B, C, D, E, F, and G—after 20, 60 and 70 minutes, 2, 4 and 8 hours of incubation, respectively.

FIG. 2 is a three-dimensional visualization of Cy3-IgGₕᵤₘ-S-S-(37-72)Tat uptake by live CH(R)-B30 cells demonstrating the internalization of the conjugate by the cell. A—an horizontal section through the cell parallel to the substrate, B and C—two perpendicular vertical sections taken in the planes indicated by the straight lines.

FIG. 3 shows uptake of PDPH-conjugated (37-72)Tat-S-S-Ox49-Alexa568 by live CH(R)-B30 cells and comparison of conjugate concentrations. A, 0.5 μM conjugate, B, 0.033 μM conjugate, C, 0.033 μM IgG-Alexa568 not conjugated with (37-72)Tat.

FIG. 4 depicts flow cytometry analysis graphs of Calcein-AM extrusion from drug resistant CH(R)B30 cells.

FIG. 5—demonstrates the effect of C219-S-S-(37-72)Tat conjugate on Calcein AM uptake by drug-resistant HU-2 cells.

FIG. 6—C219-S-S-(37-72)Tat conjugate increases the sensitivity of drug resistant HU-2 cells to Adriamycin and colchicine. 6A. Light microscopy of treated and control cells. 6B. a graph indicating percent viable cells in the presence of each drug treated with the conjugate.

### DETAILED DESCRIPTION OF THE INVENTION

The present invention is exemplified by the non-limiting example of a conjugate comprising a C219 anti-MDR antibody linked through a disulfide bridge to a translocator peptide. It is shown for the first time that such conjugates can enter cells and reduce the biological activity of membrane-bound MDR antigens, as demonstrated herein with the C219-S-S-(37-72)Tat conjugate. As demonstrated, when contacting MDR cells with said conjugate it was taken up by the MDR cells and reduced the activity of the MDR protein P-glycoprotein (P-gp).

The effect of the conjugate of the invention was unexpected as the MDR proteins to which the conjugate bind intracellularly are embedded in the lipid plasma membrane (from inside, i.e. the cytosolic compartment), of the cell and thus have almost always a three-dimensional structure that is greatly different from the spatial structure it has in solution. In addition, a mobility restriction of an antigen may considerably interfere with the neutralizing antigen-antibody reaction. In an example of the present invention, Tat fragment serving as a permeability moiety, was bound to the antibody's carbohydrate moiety and surprisingly this linkage did not interfere with the immune reactivity of the antibody.

MDR activity refers to resistance of cells, particularly, malignant cells, to either a single drug or a number of drugs which may be structurally and mechanically unrelated (cross resistance). Resistance to anti-cancer drugs may result from expression of ATP-dependent efflux pumps with broad drug specificity. These pumps belong to a family of ATP-binding cassette (ABC) transporters that share sequence and structural homology. The human ABC genes have been identified and divided into seven distinct subfamilies (ABCA-ABCG) on the basis of their sequence homology and domain organization. Resistance results because increased drug efflux lowers intracellular drug concentrations. Drugs that are affected by classical MDR include, without being limited thereto, the Vinca alkaloids (e.g. vinblastine and vincristine), the anthracylines (e.g. doxorubicin and daunorubicin), RNA transcription inhibitor actinomycin-D and the microtubule stabilizing drug paclitaxel.

It should be noted that the scope of the present invention includes not only the different classes of ABC transporters involved in drug resistance, but also other membranous proteins that extend their loops into the cytoplasm (and maybe less into the extracellular space). If such a protein has an energy requiring function (the MDR proteins that are energy-requiring pumps) the energy has to be provided from inside the cell by energy-rich molecules (ATP for MDR), and the energy-rich molecules have to be bound to binding sites of energy-requiring membrane molecules. Such binding sites are located on loops protruding into the cytoplasm. The blockage of the binding site can therefore not be achieved with substances acting from “outside”, but only from inside with a blocking substance that have been translocated into the cytoplasm.

P-gp, among other MDR proteins, is expressed within the BBB, probably as the normal mechanism of inhibiting molecules penetration through the BBB into the brain. The conjugate can be used to temporarily “open” the Blood-Brain-Barrier by impairing P-gp that is normally expressed at high levels in this barrier, thus enhancing the transport of chemotherapeutic agents (or other molecules), administered simultaneously or at a timing where the P-gp are still not active, into the brain and subsequently impairing growth of brain tumors. Without wishing to be bound by theory it is believed that the enhanced transport is achieved by the inhibition of the P-gp pump that allowing higher level of transport of medicinal agents that would have otherwise been expelled through the blood brain barrier. Typically the medicinal agent is an agent which does not pass the BBB in large amounts and may be used for diagnostic or therapeutic purposes. A preferred example of a medicinal agent is a chemotherapeutic agent used to treat brain tumors. It should be noted that the brain tumors are not necessarily MDR, as even no-MDR tumors are typically unresponsive to systemic administration until the BBB is made more available for transport.

The term “resistance” includes resistance resulting from reduced drug uptake. Non-limiting examples of drugs affected by this mechanism include the antifolate methotrexate, nucleotide analogues, such as 5-fluorouracil and 8-azaguanine and cisplatin. Resistance according may result from activation of co-ordinately regulated detoxifying systems, such as DNA repair and the cytochrome P450 mixed function oxidases. Further, resistance may result from defective apoptotic pathways or from increased activity of the ATP-dependent efflux pumps. As should be appreciated by those versed in the art, MDR may result from one or a combination of any of the above mechanisms.

There are different types of proteins which confer cells with multi drug resistance. Thus, according to the invention inhibition of MDR activity denotes inhibition by one or a combination of any of the above mechanisms in cells treated with the conjugate of the invention, thus resulting in an increase in intracellular drug concentration (as compared to the concentration thereof in non-treated MDR cells).

According to the invention such proteins include, without being limited thereto, ABC transporters known to confer drug resistance, including MDR1 (ABCB1), MRP4 (ABCC4), MRP5 (ABCC5), MRP1 (ABCC1), MRP2 (ABCC2), MRP3 (ABCC3), MXR/BCRP/ABC-p (ABCG2) all of which and more are described in detail by Gottesman et al. Preferably the MDR is the ABC transporter MDR1 (ABCB1, P-gp).

Anti-MDR antibody according to the invention denotes antibodies of any of the classes IgG, IgM, IgD, IgA, and IgE antibody capable of binding to MDR protein. The definition includes polyclonal antibodies or monoclonal antibodies. This term refers to whole antibodies or fragments of the antibodies comprising the antigen-binding domain of the antibodies, e.g. scFv, Fab, F(ab′)2, other antibodies without the Fc portion, single chain antibodies, diabodies, other fragments consisting of essentially only the variable, antigen-binding domain/fragment of the antibody, etc., which substantially retain the antigen-binding characteristics of the whole antibody from which they were derived.

The antibodies of the invention are such which specifically bind to intracellular epitopes of the MDR protein as well as antigenic binding fragments of such antibodies substantially retaining the antigen-binding characteristics of these antibodies. Antibodies binding to an antigenic epitope bound by such antibodies, as well as antibodies which bind to an antigen to which any one of the above Abs specifically bind are also within the scope of the invention. The term “substantially retain” should be understood to mean that the binding affinity of the antibody fragment for the product as determined by any of the methods mentioned below is at least 50% of the binding affinity of the whole antibody for the same variant product.

The antibodies of the invention may be produced by hybridoma cell lines (e.g. Kohler, G. and Milstein, C. 1975) or the EBV-hybridoma technique described in Cole et al., or by recombinant genetic methods well known to a person skilled in the art. The antibody may be animal derived, typically a mouse antibody as well as a human antibody, a chimeric antibody, a “humanized antibody”, a primatized antibody, etc.

Additionally, wherein the antibodies are recombinantly produced, techniques developed for production of chimeric antibodies or humanized antibodies such as those described in Morrison et al., 1984 may also be used.

Antibodies may also be produced by inducing in vivo production in the appropriate lymphocyte population or by screening recombinant immunoglobulin libraries in accordance with known methods which are described, for example, in Orlandi et al. Proc. Natl. Acad. Sci. 86:3833, (1989).

Antibodies may also be produced by DNA immunization with plasmids containing an antigenic coding sequence. Such DNA immunizing methods are described, for example, in Annu. Rev. Immunol., 15:617, (1997).

According to one embodiment, the antibodies of the invention are polyclonal antibodies produced against the intracellular domain(s) of MDR protein (in particular MDR1) or against synthetic peptides mimicking some of the intracellular fragments. According to a particular embedment, the polyclonal antibodies are raised against the (596-636) MDR1 fragment having the sequence: VRNADVIAGFDDGVIVEKGNHDELMKEKGIYFKLVTMQTAGNEVE described in Bruggemann et al. 1991. BioTechniques 10, 202-204, 206, 208-209 (1991).

Antibodies may also be produced against synthetic antigenic moieties of the MDR proteins. One example of a synthetic peptide mimicking the MDR1 protein is a peptide which corresponds to the partial sequence 592V-636E of the human P-gp, and which was synthesized with a Cys attached (for maleimide coupling) at the C-terminus (Ueda K, et al. 1987).

“Permeability” refers to the ability of an agent or substance to penetrate, pervade, or diffuse through a barrier, membrane, particularly cells' membrane, or a skin layer. Any conjugate which succeeds in penetrating into the cells whether by a passive diffusion (e.g., lipophilic moieties that penetrate the lipid bilayer of the cells), or a passive mechanist (e.g., encapsulation or liposome uptake or the like), or by active uptake (e.g. attachment to a moiety that is transported into the cells or through the membrane), is included within the scope of the present invention.

A “cell entering moiety”, denoted also “a cell permeability enhancing moiety”, according to the invention may be any moiety biological or chemical (natural, semi-synthetic or synthetic) capable of facilitating or enhancing entry of the antibody to which it is conjugated, into the target cells. Non-limiting examples of cell entering moieties include hydrophobic moieties such as lipids, fatty acids, steroids and bulky aromatic or aliphatic compounds; moieties which may have cell-membrane receptors or carriers, such as steroids, vitamins and sugars, natural and non-natural amino acids and transporter peptides. More specific examples include cationic protein transduction domains (PTDs) such as HIV-1 TAT, Drosophila Antennapedia, poly-arginine (R7) (synthetic), PTD-5 (synthetic), amphipathic PTDs such as transportan (chimeric, galanin fragment plus mastoparan), KALA and more, as described by Kabouridis et al. Other examples are small organic molecules, notably lipophilic that are known to promote transfer across cell membranes of agents that are complexed or covalently attached to them. A cell entering moiety comprising HIV-1(37-72) Tat fragment (Fawell et al. 1994) is demonstrated herein as an example for a translocation peptide.

Non-limiting examples for lipidic moieties which may be used according to the present invention: Lipofectamine, Transfectace, Transfectam, Cytofectin, DMRIE, DLRIE, GAP-DLRIE, DOTAP, DOPE, DMEAP, DODMP, DOPC, DDAB, DOSPA, EDLPC, EDMPC, DPH, TMADPH, CTAB, lysyl-PE, DC-Cho, -alanyl cholesterol; DCGS, DPPES, DCPE, DMAP, DMPE, DOGS, DOHME, DPEPC, Pluronic, Tween, BRIJ, plasmalogen, phosphatidylethanolamine, phosphatidylcholine, glycerol-3-ethylphosphatidylcholine, dimethyl ammonium propane, trimethyl ammonium propane, diethylammonium propane, triethylammonium propane, dimethyldioctadecylammonium bromide, a sphingolipid, sphingomyelin, a lysolipid, a glycolipid, a sulfatide, a glycosphingolipid, cholesterol, cholesterol ester, cholesterol salt, oil, N-succinyldioleoylphosphatidylethanolamine, 1,2-dioleoyl-sn-glycerol, 1,3-dipalmitoyl-2-succinylglycerol, 1,2-dipalmitoyl-sn-3-succinylglycerol, 1-hexadecyl-2-palmitoylglycerophosphatidylethanolamine, palmitoylhomocystiene, N,N′-Bis (dodecyaminocarbonylmethylene)-N,N′-bisq-N,N,N-trimethylammoniumethyl-ami nocarbonylmethylene)ethylenediamine tetraiodide; N,N″-Bis(hexadecylaminocarbonylmethylene)-N,N′,N″-tris((-N,N,N-trimethylammonium-ethylaminocarbonylmethylenediethylenetriamine hexaiodide; N,N′-Bis (dodecylaminocarbonylmethylene)-N,N″-bis((-N,N,N-trimethylammonium ethylaminocarbonylmethylene)cyclohexylene-1,4-diamine tetraiodide; 1,7,7-tetra-((-N,N,N,N-tetramethylammoniumethylamino-carbonylmethylene)-3-hexadecylaminocarbonyl-methylene-1,3,7-triaazaheptane heptaiodide; N,N,N′,N′-tetra((-N,N,N-trimethylammonium-ethylaminocarbonylmethylene)-N′-(1,2-dioleoylglycero-3-phosphoethanolamino carbonylmethylene)diethylenetriamine tetraiodide; dioleoylphosphatidylethanolamine, a fatty acid, a lysolipid, phosphatidylcholine, phosphatidylethanolamine, phosphatidylserine, phosphatidylglycerol, phosphatidylinositol, a sphingolipid, a glycolipid, a glucolipid, a sulfatide, a glycosphingolipid, phosphatidic acid, palmitic acid, stearic acid, arachidonic acid, oleic acid, a lipid bearing a polymer, a lipid bearing a sulfonated saccharide, cholesterol, tocopherol hemisuccinate, a lipid with an ether-linked fatty acid, a lipid with an ester-linked fatty acid, a polymerized lipid, diacetyl phosphate, stearylamine, cardiolipin, a phospholipid with a fatty acid of 6-8 carbons in length, a phospholipid with asymmetric acyl chains, 6-(5-cholesten-3b-yloxy)-1-thio-b-D-galactopyranoside, digalactosyldiglyceride, 6-(5-cholesten-3b-yloxy)hexyl-6-amino-6-deoxy-1-thio-b-D-galactopyranoside, 6-(5-cholesten-3b-yloxy)hexyl-6-amino-6-deoxyl-1-thio-a-D-mannopyranoside, 12-(((7′-diethylamino-coumarin-3-yl)carbonyl)methylamino)-octadecanoic acid; N-[12-(((7′-diethylaminocoumarin-3-yl)carbonyl)methyl-amino) octadecanoyl]-2-aminopalmitic acid; cholesteryl)4′-trimethyl-ammonio)butanoate; N-succinyldioleoyl-phosphatidylethanolamine; 1,2-dioleoyl-sn-glycerol; 1,2-dipalmitoyl-sn-3-succinyl-glycerol; 1,3-dipalmitoyl-2-succinylglycerol, 1-hexadecyl-2-palmitoylglycero-phosphoethanolamine, and palmitoylhomocysteine.

The antibody or fragment thereof according to the invention may be conjugated by any known means, for example via its amino, carboxy, S—S groups or via the Fc polysaccharide moieties. The conjugation between the antibody and the cell entering moiety may also involve a linker. Suitable linkers are known in the art (for example Hermanson, G. T. 1996). Preferably the linker is of the type that can be cleaved by intracellular enzymes this separating the antibody or its fragment from the cell entering moiety. According to one specific embodiment, the linker is a disulfide bridge, such as that described by Stein S, et al. (FEBS LETTERS 1999; 456:383-86).

In the specification and in the claims the term “linker” denotes any chemical compound, which may be present between the permeability enhancing moiety and the peptide. Preferably, the linker may be cleaved from the peptide by chemical means, by enzymatic means, or may decompose spontaneously. The linker may be pharmacologically inert or may itself provide added beneficial pharmacological activity. The term “spacer” denoting a moiety used to allow distance between the permeability-enhancing moiety and the peptide, may also be used interchangeably as a synonym for linker.

The linker may optionally comprise a protease specific cleavable sequence. A “Protease specific cleavable sequence” denotes any peptide sequence which comprises a peptide bond cleavable by a specific protease, which is more abundant within or in proximity to the malignant cells. Non-limiting examples for protease specific cleavable sequence are described in WO 02/020715. Typically a protease specific cleavable sequence includes peptides of from about two to about fourteen amino acids comprising at least one site that is cleaved by a specific protease. More preferred are peptide sequences comprising from about three to about twelve amino acids.

Non-limiting examples for specific biodegradable sequences that are degraded by proteases that are more abundant within or in proximity to the malignant cells are: Matrix metalloproteinases (for example collagenases, gelatinases and stromelysins); Aspartic proteases (for example cathepsin D, cathepsin E, pepsinogen A, pepsinogen C, rennin); Serine proteases (for example plasmin, tissue-type plasminogen activator (tPA), urokinase-type plasminogen activator (uPA); cysteine proteases (for example cathepsin B, cathepsin L, cathepsin S); asparaginyl proteases (for example legumain).

The term “antibody” is used in the broadest sense and includes monoclonal antibodies (including full length or intact monoclonal antibodies), polyclonal antibodies, multivalent antibodies, multispecific antibodies (e.g., bispecific antibodies), and antibody fragments so long as they exhibit the desired biological activity. The antibody used in conjugates according to the present invention is a molecule comprising at least the antigen-binding portion of an antibody. Further included within the scope of the invention are conjugates comprising chimeric antibodies; human and humanized antibodies; recombinant and engineered antibodies, and fragments thereof. Furthermore, the DNA encoding the variable region of the antibody can be inserted into the DNA encoding other antibodies to produce chimeric antibodies. Single chain antibodies also fall within the scope of the present invention.

“Antibody fragments” comprise only a portion of an intact antibody, generally including an antigen binding site of the intact antibody and thus retaining the ability to bind antigen. Examples of antibody fragments encompassed by the present definition include: (i) the Fab fragment, having VL, CL, VH and CH1 domains; (ii) the Fab′ fragment, which is a Fab fragment having one or more cysteine residues at the C-terminus of the CH1 domain; (iii) the Fd fragment having VH and CH1 domains; (iv) the Fd′ fragment having VH and CH1 domains and one or more cysteine residues at the C-terminus of the CH1 domain; (v) the Fv fragment having the VL and VH domains of a single arm of an antibody; (vi) the dAb fragment (Ward et al., Nature 341, 544-546 (1989)) which consists of a VH domain; (vii) isolated CDR regions; (viii) F(ab′)₂ fragments, a bivalent fragment including two Fab′ fragments linked by a disulphide bridge at the hinge region; (ix) single chain antibody molecules (e.g. single chain Fv; scFv) (Bird et al., Science 242:423-426 (1988); and Huston et al., PNAS (USA) 85:5879-5883 (1988)); (x) “diabodies” with two antigen binding sites, comprising a heavy chain variable domain (VH) connected to a light chain variable domain (VL) in the same polypeptide chain (see, e.g., EP 404,097; WO 93/11161; and Hollinger et al., Proc. Natl. Acad. Sci. USA, 90:6444-6448 (1993)); (xi) “linear antibodies” comprising a pair of tandem Fd segments (VH-CH1-VH-CH1) which, together with complementary light chain polypeptides, form a pair of antigen binding regions (Zapata et al. Protein Eng. 8(10):1057-1062 (1995); and U.S. Pat. No. 5,641,870).

Single chain antibodies can be single chain composite polypeptides having antigen binding capabilities and comprising amino acid sequences homologous or analogous to the variable regions of an immunoglobulin light and heavy chain i.e. linked V_{H}-V_{L} or single chain Fv (scFv).

A “neutralizing antibody” as used herein refers to a molecule having an antigen-binding site to a specific receptor or ligand target capable of reducing or inhibiting (blocking) activity or signaling through a receptor, as determined by in vivo or in vitro assays, as per the specification.

A “monoclonal antibody” or “mAb” is a substantially homogeneous population of antibodies to a specific antigen. Monoclonal antibodies are highly specific, being directed against a single antigen. Furthermore, in contrast to polyclonal antibody preparations that typically include different antibodies directed against different determinants (epitopes), each monoclonal antibody is directed against a single determinant on the antigen. The modifier “monoclonal” is not to be construed as requiring production of the antibody by any particular method. For example, the monoclonal antibodies to be used in accordance with the present invention may be made by the hybridoma method first described by Kohler et al., Nature 256:495 (1975), or may be made by recombinant DNA methods (see, e.g., U.S. Pat. No. 4,816,567). The “monoclonal antibodies” may also be isolated from phage antibody libraries using the techniques described in Clackson et al., Nature 352:624-628 (1991) or Marks et al., J. Mol. Biol. 222:581-597 (1991), for example.

The mAbs used in conjugates of the present invention may be of any immunoglobulin class including IgG, IgM, IgE, IgA, and any subclass thereof. A hybridoma producing a mAb may be cultivated in vitro or in vivo. High titers of mAbs can be obtained in vivo production where cells from the individual hybridomas are injected intraperitoneally into pristine-primed Balb/c mice to produce ascites fluid containing high concentrations of the desired mAbs. mAbs of isotype IgM or IgG may be purified from such ascites fluids, or from culture supernatants, using column chromatography methods well known to those of skill in the art.

The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al., Proc. Natl. Acad. Sci. USA 81:6851-6855 (1984)). Antibodies which have variable region framework residues substantially from human antibody (termed an acceptor antibody) and complementarity determining regions substantially from a mouse antibody (termed a donor antibody) are also referred to as humanized antibodies. Chimeric antibodies are primarily used to reduce immunogenicity in application and to increase yields in production, for example, where murine mAbs have higher yields from hybridomas but higher immunogenicity in humans, such that human/murine chimeric mAbs are used. In addition, complementarity determining region (CDR) grafting may be performed to alter certain properties of the antibody molecule including affinity or specificity. A non-limiting example of CDR grafting is disclosed in U.S. Pat. No. 5,225,539.

“Humanized” forms of non-human (e.g., murine) antibodies are chimeric antibodies that contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin sequence. The humanized antibody optionally will also comprise at least a portion of an immunoglobulin constant region (Fc), typically that of a human immunoglobulin. Human antibodies can be produced using various techniques known in the art. In one embodiment, the human antibody is selected from a phage library, where that phage library expresses human antibodies (Vaughan et al. Nature Biotechnology 14:309-314 (1996): Sheets et al. PNAS (USA) 95:6157-6162 (1998)); Hoogenboom and Winter, J. Mol. Biol., 227:381 (1991); Marks et al., J. Mol. Biol., 222:581 (1991)). Human antibodies can also be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al, Bio/Technology 10: 779-783 (1992); Lonberg et al., Nature 368:856-859 (1994); Morrison, Nature 368:812-13 (1994); Fishwild et al., Nature Biotechnology 14:845-51 (1996); Neuberger, Nature Biotechnology 14:826 (1996); Lonberg and Huszar, Intern. Rev. Immunol. 13:65-93 (1995). Alternatively, the human antibody may be prepared via immortalization of human B lymphocytes producing an antibody directed against a target antigen (such B lymphocytes may be recovered from an individual or may have been immunized in vitro). See, e.g., Cole et al., Monoclonal Antibodies and Cancer Therapy, Alan R. Liss, p. 77 (1985); Boerner et al., J. Immunol., 147 (1):86-95 (1991); and U.S. Pat. No. 5,750,373.

By the term “single chain variable fragment (scFv)” is meant a fusion of the variable regions of the heavy and light chains of immunoglobulin, linked together with a short (usually serine, glycine) linker. Single chain antibodies can be single chain composite polypeptides having antigen binding capabilities and comprising amino acid sequences homologous or analogous to the variable regions of an immunoglobulin light and heavy chain (linked V_{H}-V_{L} or single chain Fv (scFv)). Both V_{H} and V_{L} may copy natural monoclonal antibody sequences or one or both of the chains may comprise a CDR-FR construct of the type described in U.S. Pat. No. 5,091,513, the entire contents of which are incorporated herein by reference. The separate polypeptides analogous to the variable regions of the light and heavy chains are held together by a polypeptide linker. Methods of production of such single chain antibodies, particularly where the DNA encoding the polypeptide structures of the V_{H} and V_{L} chains are known, may be accomplished in accordance with the methods described, for example, in U.S. Pat. Nos. 4,946,778, 5,091,513 and 5,096,815, the entire contents of each of which are incorporated herein by reference.

A “molecule having the antigen-binding portion of an antibody” as used herein is intended to include not only intact immunoglobulin molecules of any isotype and generated by any animal cell line or microorganism, but also the antigen-binding reactive fraction thereof, including, but not limited to, the Fab fragment, the Fab′ fragment, the F(ab′)₂ fragment, the variable portion of the heavy and/or light chains thereof, Fab miniantibodies (see WO 93/15210, U.S. patent application Ser. No. 08/256,790, WO 96/13583, U.S. patent application Ser. No. 08/817,788, WO 96/37621, U.S. patent application Ser. No. 08/999,554, the entire contents of which are incorporated herein by reference), dimeric bispecific miniantibodies (see Muller et al., 1998) and chimeric or single-chain antibodies incorporating such reactive fraction, as well as any other type of molecule or cell in which such antibody reactive fraction has been physically inserted, such as a chimeric T-cell receptor or a T-cell having such a receptor, or molecules developed to deliver therapeutic moieties by means of a portion of the molecule containing such a reactive fraction. Such molecules may be provided by any known technique, including, but not limited to, enzymatic cleavage, peptide synthesis or recombinant techniques.

Antibodies can be obtained by administering the antigen, or epitope-bearing fragments, analogs, or cells expressing, to an animal, preferably a nonhuman, using routine protocols.

For preparation of monoclonal antibodies, any technique known in the art that provides antibodies produced by continuous cell line cultures can be used. Examples include various techniques, such as those in Kohler, G. and Milstein, C., Nature 256: 495-497 (1975); Kozbor et al., Immunology Today 4: 72 (1983); Cole et al., pg. 77-96 in MONOCLONAL ANTIBODIES AND CANCER THERAPY, Alan R. Liss, Inc. (1985).

Besides the conventional method of raising antibodies in vivo, antibodies can be generated in vitro using phage display technology. Such a production of recombinant antibodies is much faster compared to conventional antibody production and they can be generated against an enormous number of antigens. Furthermore, when using the conventional method, many antigens prove to be non-immunogenic or extremely toxic, and therefore cannot be used to generate antibodies in animals. Moreover, affinity maturation (i.e., increasing the affinity and specificity) of recombinant antibodies is very simple and relatively fast. Finally, large numbers of different antibodies against a specific antigen can be generated in one selection procedure. To generate recombinant monoclonal antibodies one can use various methods all based on display libraries to generate a large pool of antibodies with different antigen recognition sites. Such a library can be made in several ways: One can generate a synthetic repertoire by cloning synthetic CDR3 regions in a pool of heavy chain germline genes and thus generating a large antibody repertoire, from which recombinant antibody fragments with various specificities can be selected. One can use the lymphocyte pool of humans as starting material for the construction of an antibody library. It is possible to construct naive repertoires of human IgM antibodies and thus create a human library of large diversity. This method has been widely used successfully to select a large number of antibodies against different antigens. Protocols for bacteriophage library construction and selection of recombinant antibodies are provided in the well-known reference text Current Protocols in Immunology, Colligan et al (Eds.), John Wiley & Sons, Inc. (1992-2000), Chapter 17, Section 17.1. Alternatively, phage display technology can be utilized to select antibody genes with binding activities towards a polypeptide of the invention either from repertoires of PCR amplified v-genes of lymphocytes from humans screened for possessing anti-VEGF or from libraries (McCafferty, et al., (1990), Nature 348, 552-554; Marks, et al., (1992) Biotechnology 10, 779-783). The affinity of these antibodies can also be improved by, for example, chain shuffling (Clackson et al., (1991) Nature 352:628).

Anti-idiotype antibodies specifically immunoreactive with an antibody of the invention are also comprehended.

Techniques for the production of single chain antibodies (U.S. Pat. No. 4,946,778) can be adapted to produce single chain antibodies to polypeptides or polynucleotides of this invention. Also, transgenic mice, or other organisms such as other mammals, can be used to express humanized antibodies immunospecific to the polypeptides or polynucleotides of the invention.

The term “therapeutically effective amount” refers to an amount of a drug effective to treat a disease or disorder in a mammal. In the case of cancer, the therapeutically effective amount of the drug may reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the disorder. To the extent the drug may prevent growth and/or kill existing cancer cells, it may be cytostatic and/or cytotoxic. For cancer therapy, efficacy in vivo can, for example, be measured by assessing the duration of survival, time to disease progression (TTP), the response rates (RR), duration of response, and/or quality of life.

Administration of a therapeutically active amount of pharmaceutical compositions of the present invention is defined as an amount effective, at dosages and for periods of time necessary to achieve the desired result. For example, a therapeutically active amount of a substance may vary according to factors such as the disease state, age, sex, and weight of the individual, and the ability of the substance to elicit a desired response in the individual. Dosage regimes may be adjusted to provide the optimum therapeutic response. For example, several divided doses may be administered daily or the dose may be proportionally reduced as indicated by the exigencies of the therapeutic situation.

The terms “cancer” and “cancerous” refer to or describe the physiological condition in mammals that is typically characterized by unregulated cell growth. Examples of cancer include but are not limited to, carcinoma, lymphoma, blastoma, sarcoma, and leukemia. More particular examples of such cancers include squamous cell cancer, lung cancer (including small-cell lung cancer, non-small cell lung cancer, adenocarcinoma of the lung, and squamous carcinoma of the lung), cancer of the peritoneum, hepatocellular cancer, gastric or stomach cancer (including gastrointestinal cancer), pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney or renal cancer, liver cancer, prostate cancer, vulval cancer, thyroid cancer, hepatic carcinoma and various types of head and neck cancer, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia); chronic lymphocytic leukemia (CLL); acute lymphoblastic leukemia (ALL); Hairy cell leukemia; chronic myeloblastic leukemia; and post-transplant lymphoproliferative disorder (PTLD), as well as abnormal vascular proliferation associated with phakomatoses, edema (such as that associated with brain tumors), and Meigs' syndrome.

The term “anti-neoplastic composition” refers to a composition useful in treating cancer comprising at least one active therapeutic agent capable of inhibiting or preventing tumor growth or function, and/or causing destruction of tumor cells. Therapeutic agents suitable in an anti-neoplastic composition for treating cancer include, but not limited to, chemotherapeutic agents, radioactive isotopes, toxins, cytokines such as interferons, and antagonistic agents targeting cytokines, cytokine receptors or antigens associated with tumor cells. For example, therapeutic agents useful in the present invention can be antibodies such as anti-HER2 antibody and anti-CD20 antibody, or small molecule tyrosine kinase inhibitors such as VEGF receptor inhibitors and EGF receptor inhibitors. Preferably the therapeutic agent is a chemotherapeutic agent.

A “chemotherapeutic agent” is a chemical compound useful in the treatment of cancer. Examples of chemotherapeutic agents include alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gamma1I and calicheamicin omegaI1 (e.g., Agnew, Chem. Intl. Ed. Engl. 33:183-186 (1994)); dynemicin, including dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN® doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′, 2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL® paclitaxel (Bristol-Myers Squibb Oncology, Princeton, N.J.), ABRAXANE™ Cremophor-free, albumin-engineered nanoparticle formulation of paclitaxel (American Pharmaceutical Partners), and TAXOTERE® doxetaxel (Rhone-Poulenc Rorer, Antony, France); chloranbucil; GEMZAR® gemcitabine; 6-thioguanine; mercaptopurine; methotrexate; platinum coordination complexes such as cisplatin, oxaliplatin and carboplatin; vinblastine; platinum; etoposide (VP-16); ifosfamide; mitoxantrone; vincristine; NAVELBINE® vinorelbine; novantrone; teniposide; edatrexate; daunomycin; aminopterin; xeloda; ibandronate; irinotecan (e.g., CPT-11); topoisomerase inhibitor RFS 2000; difluoromethylornithine (DMFO); retinoids such as retinoic acid; capecitabine; and pharmaceutically acceptable salts, acids or derivatives of any of the above.

Also included in this definition are anti-hormonal agents that act to regulate or inhibit hormone action on tumors such as anti-estrogens and selective estrogen receptor modulators (SERMs), including, for example, tamoxifen (including NOLVADEX® tamoxifen), raloxifene, droloxifene, 4-hydroxytamoxifen, trioxifene, keoxifene, LY117018, onapristone, and FARESTON toremifene; aromatase inhibitors that inhibit the enzyme aromatase, which regulates estrogen production in the adrenal glands, such as, for example, 4(5)-imidazoles, aminoglutethimide, MEGASE® megestrol acetate, AROMASIN® exemestane, formestanie, fadrozole, RIVISOR® vorozole, FEMARA® letrozole, and ARIMIDEX® anastrozole; and anti-androgens such as flutamide, nilutamide, bicalutamide, leuprolide, and goserelin; as well as troxacitabine (a 1,3-dioxolane nucleoside cytosine analog); antisense oligonucleotides, particularly those which inhibit expression of genes in signaling pathways implicated in abherant cell proliferation, such as, for example, PKC-alpha, Ralf and H-Ras; ribozymes such as a VEGF expression inhibitor (e.g., ANGIOZYME® ribozyme) and a HER2 expression inhibitor; vaccines such as gene therapy vaccines, for example, ALLOVECTIN® vaccine, LEUVECTIN® vaccine, and VAXID® vaccine; PROLEUKIN® mL-2; LURTOTECAN® topoisomerase 1 inhibitor; ABARELIX® rmRH; and pharmaceutically acceptable salts, acids or derivatives of any of the above.

### Pharmacology

The molecules of the present invention as active ingredients are dissolved, dispersed or admixed in an excipient that is pharmaceutically acceptable and compatible with the active ingredient as is well known. Suitable excipients are, for example, water, saline, phosphate buffered saline (PBS), dextrose, glycerol, ethanol, or the like and combinations thereof. Other suitable carriers are well known to those skilled in the art. (for example, Ansel et al., 1990 and Gennaro, 1990). In addition, if desired, the composition can contain minor amounts of auxiliary substances such as wetting or emulsifying agents, pH buffering agents.

In such pharmaceutical and medicament formulations, the active agent is preferably utilized together with one or more pharmaceutically acceptable carrier(s) and optionally any other therapeutic ingredients. The carrier(s) must be pharmaceutically acceptable in the sense of being compatible with the other ingredients of the formulation and not unduly deleterious to the recipient thereof. The active agent is provided in an amount effective to achieve the desired pharmacological effect, as described above, and in a quantity appropriate to achieve the desired daily dose.

The pharmaceutical composition according to the invention can be prepared by per se known methods for. Suitable physiologically acceptable carriers are described, for example, in Remington's Pharmaceutical Sciences (Remington's Pharmaceutical Sciences, Mack Publishing Company, Easton, Pa., USA 1985) or Handbook of Pharmaceutical Additives (compiled by Michael and Irene Ash, Gower Publishing Limited, Aldershot, England (1995)). On this basis, the compositions include, albeit not exclusively, solutions of the substances in association with one or more pharmaceutically acceptable carrier or diluents, and may be contained in buffered solutions with a suitable pH and/or be iso-osmotic with physiological fluids.

Typically, the conjugates of the present invention comprising the antigen binding portion of an antibody will be suspended in a sterile saline solution for therapeutic uses. The pharmaceutical compositions may alternatively be formulated to control release of active ingredient (molecule comprising the antigen binding portion of an antibody) or to prolong its presence in a patient's system. Numerous suitable drug delivery systems are known and include, e.g., implantable drug release systems, hydrogels, hydroxymethylcellulose, microcapsules, liposomes, microemulsions, microspheres, and the like. Controlled release preparations can be prepared through the use of polymers to complex or adsorb the molecule according to the present invention. For example, biocompatible polymers include matrices of poly(ethylene-co-vinyl acetate) and matrices of a polyanhydride copolymer of a stearic acid dimer and sebaric acid (Sherwood et al, 1992). The rate of release of the molecule according to the present invention, i.e., of an antibody or antibody fragment, from such a matrix depends upon the molecular weight of the molecule, the amount of the molecule within the matrix, and the size of dispersed particles (Saltzman et al., 1989 and Sherwood et al., 1992). Other solid dosage forms are described in Ansel et al., 1990 and Gennaro, 1990.

The pharmaceutical composition of this invention may be administered by any suitable means, such as orally, topically, intranasally, subcutaneously, intramuscularly, intravenously, intra-arterially, intraarticulary, intralesionally or parenterally. Ordinarily, intravenous (i.v.), intraarticular, topical or parenteral administration will be preferred.

It will be apparent to those of ordinary skill in the art that the therapeutically effective amount of the molecule according to the present invention will depend, inter alia upon the administration schedule, the unit dose of molecule administered, whether the molecule is administered in combination with other therapeutic agents, the immune status and health of the patient, the therapeutic activity of the molecule administered and the judgment of the treating physician. As used herein, a “therapeutically effective amount” refers to the amount of a molecule required to alleviate one or more symptoms associated with a disorder being treated over a period of time.

Although an appropriate dosage of a molecule of the invention varies depending on the administration route, type of molecule (polypeptide, polynucleotide, organic molecule etc.) age, body weight, sex, or conditions of the patient, and should be determined by the physician in the end, in the case of oral administration, the daily dosage can generally be between about 0.01 mg to about 500 mg, preferably about 0.01 mg to about 50 mg, more preferably about 0.1 mg to about 10 mg, per kg body weight. In the case of parenteral administration, the daily dosage can generally be between about 0.001 mg to about 100 mg, preferably about 0.001 mg to about 10 mg, more preferably about 0.01 mg to about 1 mg, per kg body weight. The daily dosage can be administered, for example in regimens typical of 1-4 individual administration daily. Other preferred methods of administration include intraarticular administration of about 0.01 mg to about 100 mg per kg body weight. Various considerations in arriving at an effective amount are described, e.g., in Goodman and Gilman's: The Pharmacological Bases of Therapeutics, 8th ed., Pergamon Press, 1990; and Remington's Pharmaceutical Sciences, 17th ed., Mack Publishing Co., Easton, Pa., 1990.

Suitable dosing regimens of combination chemotherapies are known in the art and described in, for example, Saltz et al. (1999) Proc ASCO 18:233a and Douillard et al. (2000) Lancet 355:1041-7.

The following examples are intended to illustrate how to make and use the compounds and methods of this invention and are in no way to be construed as a limitation. Although the invention will now be described in conjunction with specific embodiments thereof, it is evident that many modifications and variations will be apparent to those skilled in the art. Accordingly, it is intended to embrace all such modifications and variations that fall within the spirit and broad scope of the appended claims.

### EXAMPLES

In the following examples, the membrane-residing multi-drug resistant protein P-gp (MDR-1) was investigated. As an a non-limiting example of the embodiments of the present invention, a conjugate comprising the monoclonal anti-P-gp antibody C219 and an HIV-1-Tat fragment of residues 37-72 (Frankel et al. 1988, Mann et al. 1991), was designed and tested demonstrating that P-gp activity can be inhibited from inside the cells using antibodies against cytoplasmic epitopes of the protein.

P-gp is an ATP-powered outward pump of lipophilic substrates which consists of four domains arranged in the sequence NH₂-TMD₁-NBD₁-linker peptide-TMD₂-NBD₂-COOH. Each hydrophobic transmembrane domain TMD has six membrane segments and binds neutral or positively charged lipophilic substrates. Each hydrophilic nuclear binding domain extends into the cytoplasm and presents one cytoplasmic ATP binding site. When a substrate binds to a TMD, the ensuing conformational change is transmitted to the NBD. In response, the ATP turnover at the NBD increases and the energy liberated by ATP-hydrolysis is fed back to the TMD. This leads to an increased outward transport of the bound substrate. C219 is a monoclonal antibody of mouse origin directed against the NBD of MDR1_CRIGR (Kartner et al. 1985). It binds to the sequence VVQAALD (565-571) in NBD₁ and to VVQEALD (1210-1216) in NBD₂ (Georges et al. 1990). The sequences overlap with the second ATP-sections in the P-loops 528 to 598 and 1123 to 1243, respectively. C219 has been shown to inhibit the ATPase activity in membrane preparations of MDR cells (Georges et al. 1991, Kokubu et al. 1997). (37-72) Tat from the HIV HV1B1 isolate is a peptide with a positive charge contributed by 11 basic amino acids (Ratner et al. 1985). The following has now been demonstrated: i. Translocated (37-72)Tat as well as IgG-(37-72)Tat conjugates can be visualized inside colchicine-resistant CHO and mouse lymphoma cells exogenously expressing MDR; and ii. The C219-S-S-(37-72)Tat conjugate reduces the activity of MDR protein activity in such cells.

### Materials and Methods

Monoclonal antibody (Mab) C219 was purchased from Signet Pathology Systems, Dedham, Mass. 02026, USA. Ox-49 Mab and human immunoglobulin were generous gifts from Prof. Alexandru Stan, Dept. of Neuropathology, Hannover Med. School and Prof. Philipp Lazarovici, Dept. of Pharmacology, The Hebrew University of Jerusalem, respectively. Ox49 is a mouse anti-rat Mab directed against surface epitopes particularly of hematopoietic cells with no obvious counterparts in other species (Paterson et al., 1987). Other drugs and chemicals included: sulfosuccinimidyl-6[3′-(2-pyridyldithio)propionamido]hexanoate (slcSPDP), sulfosuccinimidyl-4-(N-maleimido-methyl)-cyclohexane-1-carboxylate (sSMCC), N-[β-maleimidocaproic acid]hydrazide (EMCH), 3-(2-pyridyldithio)-propionyl hydrazide (PDPH) (Pierce, Rockford, Ill., USA), colchicine, dimethylformamide, Ellman's reagent, propidium iodide (Sigma-Aldrich, Taufkirchen, Germany), Cy3 (Amersham Life Sci, Arlington Heights, Ill., USA), Alexa 568 succinimidyl ester protein labeling kit, calcein-AM, and d-verapamil (Molecular Probes, Leiden, The Netherlands), trypsin, MEM alpha medium, CO₂-independent medium, fetal calf serum, penicillin+streptomycin (Gibco, Scotland branch, UK), ceftazidim (GlaxoSmithKline, Munich, Germany), sodium periodate, dimethylsulfoxide, glycerol (Merck, Darmstadt, Germany). Throughout the conjugate syntheses, Spectraphor™ tubing (Spectrum Laboratories, Rancho Dominguez, Calif., USA) with a 25 kDa cut-off was used for dialysis. All concentrations were determined from the respective extinctions at the appropriate wavelengths. The following extinction coefficients were used: E(IgG 10⁻⁵ M, 1 cm, 280 nm)=2.1; E((37-72) Tat, 10⁻⁵ M, 1 cm, 280 nm)=0.012; E(Alexa 568, 10⁻⁵ M, 1 cm, 577 nm)=0.913; E(Cy3, 10⁻⁵ M, 1 cm, 550 nm)=1.5; E(fluorescein, M, 1 cm, 494 nm)=0.62; E(pyridyl-2-thion 10⁻⁵ M, 1 cm, 343 nm)=0.0808; thiol groups with Ellman's reagent E(chromophore, 10⁻⁵ M, 1 cm, 420 nm)=0.136. The 280 nm spill-over extinctions contributed by Alexa 568, Cy3, and PDP, respectively, were calculated as follows: E(Alexa 568, 280 nm)=0.46×E(Alexa 568, 577 nm); E(Cy3, 280 nm)=0.08×E(Cy3, 550 nm); E(pyridyl-2-thion, 280 nm)=0.63×E(pyridyl-2-thion, 343 nm).

### (37-72)Tat Synthesis

The (37-72) Tat fragment of HIV-HV1B1 Tat was synthesized as previously described (Stein et al., 1999).

### Synthesis of Conjugates

The (37-72) fragment of Tat was selected for conjugate synthesis as its (37)-Cys residue has a free thiol group ready for coupling via a disulfide bond. The following immunoglobulins were linked to the (37)Cys of (37-72)Tat using the specified linkers: (1) Mab C219, linker PDPH; (2) Mab C219, linker EMCH; (3) Mab Ox49, linker PDPH; (4) Mab Ox49, linker EMCH; (5) Alexa 568-Ox49, linker PDPH; (6) Alexa 568-Ox49, linker EMCH; (7)Alexa 568-Ox49, linker slcSPDP; (8) Alexa 568-IgGₕᵤₘ, linker slcSPDP; (9) Alexa 568-IgGₕᵤₘ, linker SMCC. The non-labelled C219 and Ox49 conjugates (No. 1-4) were used in flow cytometry experiments to study their effect on MDR activity. Cy3 or Alexa 568-labelled conjugates (No. 5-9) were used to visualize their translocation into live cells with confocal microscopy. In all fluorescent conjugates, the immunoglobulin moiety was first labelled with the fluorophore, purified, and finally linked to the (37-72)Tat peptide. The conjugates (7) and (8) were synthesized as described for an F(ab′)₂ conjugate. In the conjugates (1), (2), (3), and (4) above, the Mab C219 and Mab Ox49 were linked to the Tat fragment through their polysaccharide moieties to better preserve their immune reactivity. These moieties were oxidized with sodium periodate in the dark at 0° C. As an example, the synthesis of conjugate (1) is outlined: to C219 (1.02×10⁻⁵ M in 0.1 M acetate buffer pH 5.5, 0° C.) was added sodium periodate (40 mM in acetate buffer, 0° C.) to a final concentration of 2 mM. After 30 min the oxidation was quenched with glycerol (final concentration 60 mM) for >30 min. The reaction mixture was removed from the dark, dialyzed against PBS-EDTA pH 7.0, and its protein content was determined. PDPH (0.4 M in DMSO) was added to a final concentration of 20 mM. >5 h later the reaction product was purified by dialysis against PBS-EDTA pH 7.0 and its protein content was determined. (37-72)Tat was then conjugated through thiol-disulfide exchange. For the thioether conjugates (2) and (4), (37-72)Tat peptide was reacted with an equimolar amount of EMCH, and the reaction product was conjugated to the oxidized and dialyzed Mab.

### Cells

MDR1_CRIGR, the P-gp of Chinese hamster (CHO) cells, which were used in the present experiments, consists of 1278 amino acids (Endicott et al. 1991). The CHO cell clone CH(R)B30, exogenously expressing MDR, was a gift from Prof. B. Tümmler (Hannover Medical School) (Kokubu et al., 1997). Cells were grown in alpha-MEM medium supplemented with 10% Fetal Calf Serum (FCS), penicillin 50 U/ml, streptomycin 50 μg/ml, and ceftazidim 50 μg/ml. For propagation of the clone, colchicine was added to this medium to a final concentration of 20 μg/ml. Cells to be used for experiments were then grown without colchicine and at high density to extend the half-life of ABCB1 (Tsuruo et al., 1981).

T-25 cells (Hochman et al, 1984), derived from S49 mouse lymphoma, were transduced with a retrovirus containing the human MDR1 cDNA, as previously described (Galski et al. 1995). The resultant cells (HU-2) are cross-resistant to colchicine, doxorubicin, vinblastine and actinomycin D, and their resistance to colchicine is reversed by verapamil. T-cell lymphoma derived cell lines were grown in Dulbecco's modified eagle medium (DMEM) supplemented with 10% heat-inactivated horse serum, at 37° C. in a humidified atmosphere containing 5% CO₂.

### Flow Cytometry Analysis

Cells were diluted in alpha MEM to about 1.5×10⁶ cells/ml. Volumes of 1 ml were distributed to 2 ml Eppendorf tubes. The caps used to close the tubes were punctured in their center to enable gas exchange in the incubator (5% CO₂, 95% air, 37° C.) during incubation with various substances. The capped tubes were positioned with their longitudinal axes inclined to 45° on a roller platform and were rotated in the incubator. The cells were incubated with conjugates, proteins, or peptides, respectively, at various concentrations and for various times. Separate cell samples were exposed to a single high dose of R(+)-verapamil 25 μM for 15 min to ascertain the responsiveness of the cells (Homolya et al., 1993). Calcein-AM was added to a final concentration of 1 μM or 2 μM at least 30 min before the end of incubation. Calcein-AM, a substrate of ABCB1, is a hydrophobic non-fluorescent substance that easily enters cells. Once in the cytoplasm, calcein-AM is hydrolysed, and the ensuing fluorescent calcein is retained within the cells. In drug resistant cells, calcein-AM is extruded by the ABCB1 transporter. Therefore, impairing ABCB1 activity in drug-resistant cells, exposed to Calcein-AM results in increased intracellular production and retention of fluorescent Calcein (Hollo et al. 1994, Potocky et al., 2003). To cells in separate wells, propidium iodide was added to a final concentration of 1 or 2 μg/ml 15-25 min before the end of incubation to demonstrate the viability of the cells (Young, I. T., 1977). All vials were stopped by addition of ice-cold medium and then put on ice for subsequent flow cytometry (beginning about 1 h after the end of incubation). Flow Cytometry was performed with a Becton Dickinson FACScan flow cytometer under control of the CELLQuest Pro software. The 530±15 nm bandpass filter was used for the measurement of calcein and the 585±21 nm filter for propidium iodide. The following instrument settings were determined for CH(R)B30 cells: amplifications FSC E-1, 4.51, lin; SCC 399, 1.04, lin; FL1-H 306, 1.0, log; FL2-H 332, 1.0, log; threshold FSC 100; compensations FL1-H−% FL2-H=6%, FL2-H−% FL1-H=26.7%. 100,000 cells, in a few experiments 50,000 cells per vial were counted. The WinMDI 2.8 software (Trotter J. computer program WinMDI 2.8 La Jolla, Calif.): The Scripps Research Institute; 2000. http://facs.scripps.edu/software.html) was used for the production of figures. A difference between two curves was considered significant if p was <0.001 in the Kolmogorov-Smirnov test for the analysis of histograms, and if, in addition, the difference was large enough to be biologically meaningful (Paterson 1987). For the construction of the concentration-response curves, the concentration-related medians of the fluorescence histograms and their 3σ fiducial limits were calculated as follows: using WinMDI, every histogram (a grouped frequency distribution) was saved as a tagged file (0<x<1023=channel number, y=cells per channel) which in turn was opened in Excel™. The median channel x_{M} of the y distribution and y_{M}, the number of cells in x_{M}, were determined. The 3σ values were calculated from σ=1.5*N^{0.5}/y_{M}, where N is the total number of fluorescent cells counted.²³ The fiducial limits in the channel domain were then x_{M±3σ}=x_{M}±3σ. Finally, the channel values x_{M}, x_{M+3σ}, x_{M-3σ} were converted to the relative fluorescence intensities z_{M}, z_{M+3σ}, z_{M-3σ} using the equation z=10^{x/256}, where 256 is the scaling factor for 1024 channels and 4 log decades of z. (Becton-Dickinson: BD CellQuest Pro Software User's Guide (San Jose, Calif.; 2000).

### Confocal Microscopy

A 250 μl volume of colchicine-free alpha MEM containing about 50,000 CH(R)B30 cells was pipetted into each chamber of a NUNC Lab-Tek II eight-chamber slide system. The cells were allowed 12 h to recover and attach. The alpha MEM was then exchanged against warm CO₂-independent medium containing conjugates, (37-72)Tat, or immunoglobulins at various concentrations. The cells were incubated for various times. Live cells were observed using confocal microscopy. To visualize the cell borders, substances reacting with or dissolving in cytoplasmic membranes were not used in order not to interfere with the MDR activity. A negative staining technique was used instead: just prior to imaging, Oregon Green was added to the cell culture medium to a concentration of 0.1-1 μM. The anionic Oregon Green was excluded from live cells, so that upon confocal imaging, the cell volume appeared dark. The cell borders could then be retrieved by digital image processing (edge detection). Confocal microscopy was done using a Leica TCS NT scanhead mounted on a DM IRBE inverted microscope, and a 40×/NA=1.25 oil immersion objective. Excitation was provided by an Argon-Krypton laser, using the 488 nm line to excite Oregon Green, and the 568 nm line to excite Alexa-568 and Cy3. A BP530/30 (530 nm center wavelength, 30 nm bandwidth) emission filter was used for Oregon Green, and an LP590 nm low pass emission filter was used for Alexa-568. A RSP580 dichroic mirror split the emission between the two emission channels. Although the two channels could be acquired sequentially in order to eliminate the possibility of Oregon Green emission in the Alexa-568 channel, we found that such bleedthrough was negligible under the measurement conditions employed. Therefore, in practice, both dyes were observed simultaneously. Images were processed using ImageJ (Rasband W S. 1997-2004), and Image Pro Plus (Media Cybernetics, Silver Spring, Md.). Standard image processing operations included the use of a 3×3 median filter to remove point noise, and linear contrast stretch. Specific image processing operations relevant to particular figures are described in the legend to figures.

P-gp-Dependent Efflux of Calcein AM from Cells

The non-fluorescent dye calcein AM (Molecular Probes, Leiden Netherlands) penetrates the membrane and is a substrate for the efflux carriers of P-gp, which pump calcein to AM out of the cells. Within the cell, cytoplasmic esterases release fluorescent calcein from calcein AM that has managed to enter by escaping the action of P-glycoprotein. The kinetics of calcein formation can be monitored continuously (Essodaigui et al., 1998; Essodaigui et al., 1999). When blockers inhibit the pumping activity of P-gp, more fluorescent calcein accumulates within the cells and can be assayed by its fluorescence. Cells were harvested in logarithmic growth phase and resuspended in PBS containing 10 mM glucose. 1×10⁵ cells were incubated with the desired concentration of blocker for 10 min at 37° C. or with the conjugate for 4 h at RT. Stocks of the blocker verapamil (Sigma) was prepared in DMSO. In the assay, a maximum concentration of 0.5% DMSO was used. Calcein AM was added to a final concentration of 250 nM. Fluorescence was read after 30 min at 37° C. from the bottom of the wells, with an excitation of 485 nm and an emission of 530 nm, using a microplate fluorescence reader (FL600, Biotek).

### Cell Viability

To measure the sensitivity of HU-2 cells to toxic drugs, 1×105 cells per well were incubated with 10 μg of the conjugate for 4 hrs. 2 μg/ml of Adriamycin or 1 μg/ml of colchicine was then added and the cells incubated for a further 96 hrs. Stocks were prepared in DMSO. The maximal final concentration of DMSO in the assays was 0.5% (v/v). After 96 h, the cells were checked by light microscopy and viable cells were counted by Trypan blue exclusion.

### Results

Immunoglobulin-Tat Conjugates Translocate into Drug Resistant Cells

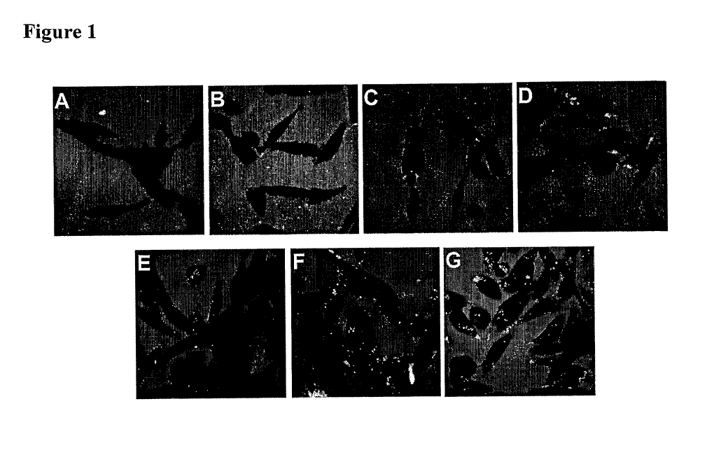

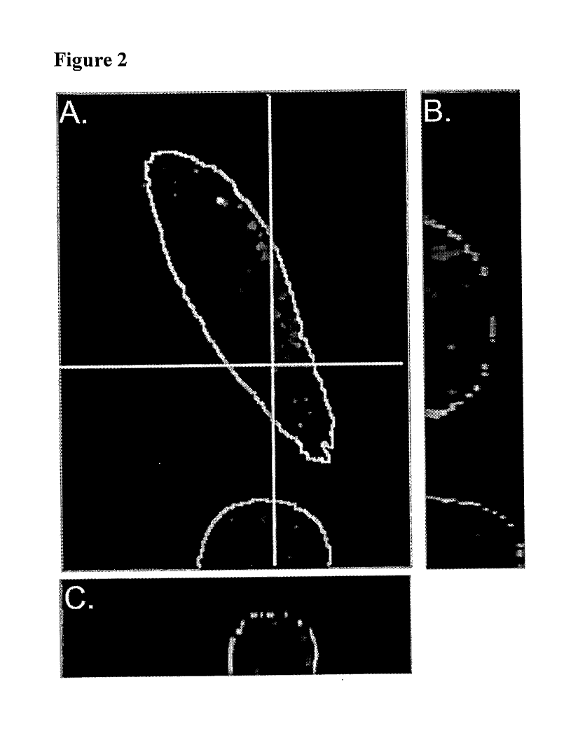

A prerequisite for functional inhibition of ABCB1 activity from within live cells is the successful translocation of Tat-conjugates into multi-drug resistant cells. Confocal microscopy was used to study the uptake of various IgG-(37-72)Tat conjugates into drug (Colchicine) resistant CH(R)B30 cells. FIG. 1, shows the time-dependent uptake of a fluorescent thioether Alexa568-IgGₕᵤₘ-S-(37-72)Tat conjugate by CH(R)B30 cells. Ch(R)B30 cells were incubated with 1 μM of the thioether conjugate (37-72)Tat-S-IgGₕᵤₘ-Alexa568. The image in A was acquired immediately after addition of the conjugate, while the images in B, C, D, E, F, and G were acquired after 20, 60 and 70 minutes, 2, 4 and 8 hours of incubation, respectively. The images are projections of sections 4-7. The system settings in A and B were identical. In C and D the detector gain was reduced to avoid saturation. The images in C and D have been stretched to normalize the gain of the Alexa-568 background to the background of A and B. The gain of the Alexa568 channel was lower for F than for E and still a bit lower for G. Therefore, absolute intensities cannot be compared. However, the trend is for longer incubation times to result in stronger signals, with a large gain difference from 2 to 4 hours, and a much smaller difference between 4 and 8 hours. Uptake was maximal within 2 hours. A similar time dependence was observed upon incubation with a disulfide Alexa568-IgGₕᵤₘ-S-S-(37-72)Tat conjugate. To ascertain the intracellular localization of conjugates, FIG. 2 demonstrates, in three dimensions, the internalization of fluorescent disulfide Cy3-IgGₕᵤₘ-S-S-(37-72)Tat conjugate in intact CH(R)B30 cells. Intact cells were incubated overnight with 0.45 μM conjugate. Fluorescein was added to the medium just prior to imaging. This provides a negative image of the cell's volume by labelling the extra cellular space. The lines show the cell surface (found by edge detection of the fluorescein image), and the punctate image shows the Cy3 labelled IgG. In addition to a horizontal section through the cell parallel to the substrate (A), two perpendicular vertical sections are shown (B and C), taken in the planes indicated by the lines. These demonstrate the internalization of the conjugate by the cell.

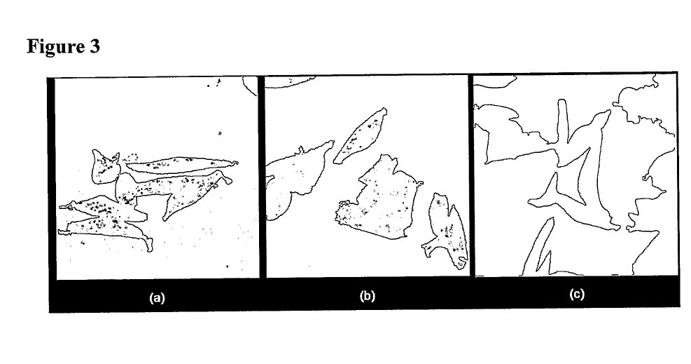

FIG. 3 demonstrates that PDPH-conjugated (37-72)Tat-S-S-Ox49-Alexa568 conjugate uptake is dose-dependent and can be visualized at a concentration of 33 nM using a mouse monoclonal antibody (Ox49) disulfide Alexa568-Ox49-S-S-(37-72)Tat conjugate. Different conjugate concentrations were incubated with intact cells for 3 hours. A, 0.5 μM conjugate, projections of sections 7-9. B, 0.033 μM conjugate, projections 4-6. C, 0.033 μM IgG-Alexa568 not conjugated with (37-72)Tat, projections 4-6. In these images, the solid black lines show the cell outline (found by edge detection of the negative stain). Point noise has been reduced through use of a 3×3 median filter, and background was estimated using an 11×11 top-hat morphological filter and subtracted from the images. The original images were obtained at different excitation intensities in order to obtain signals in the middle of the detection range of the instrument. The images shown have been normalized for differences in excitation intensity.

From the confocal analysis it is evident that both thioether and disulfide Tat conjugates are taken up by drug resistant CH(R)B30 cells, irrespective of their IgG component (whether human immunoglobulins or mouse monoclonal antibodies). Uptake can be visualized at a conjugate concentration of 33 nM and within 20 minutes after addition of the conjugates.

### Antibody-Tat Conjugates Impair ABCB1 Transporter Activity

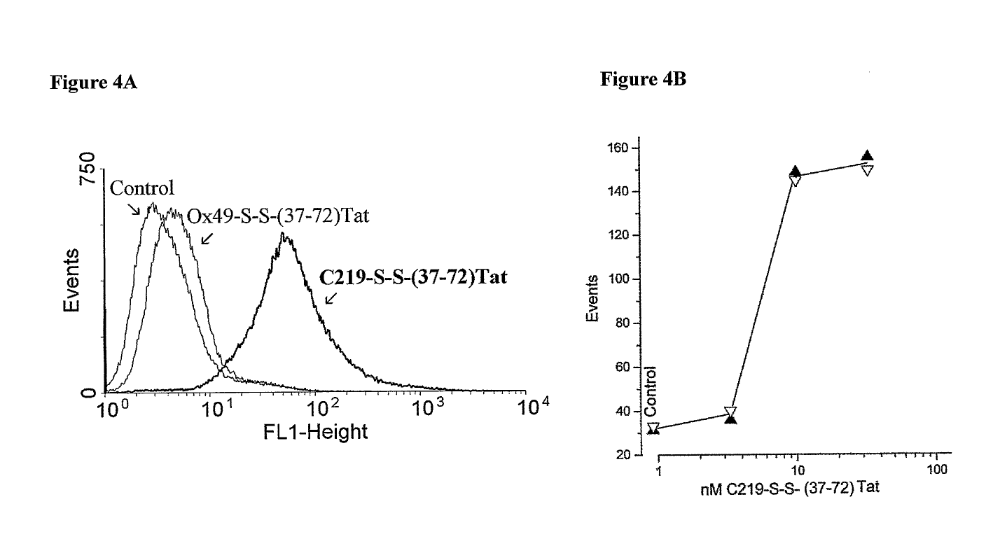

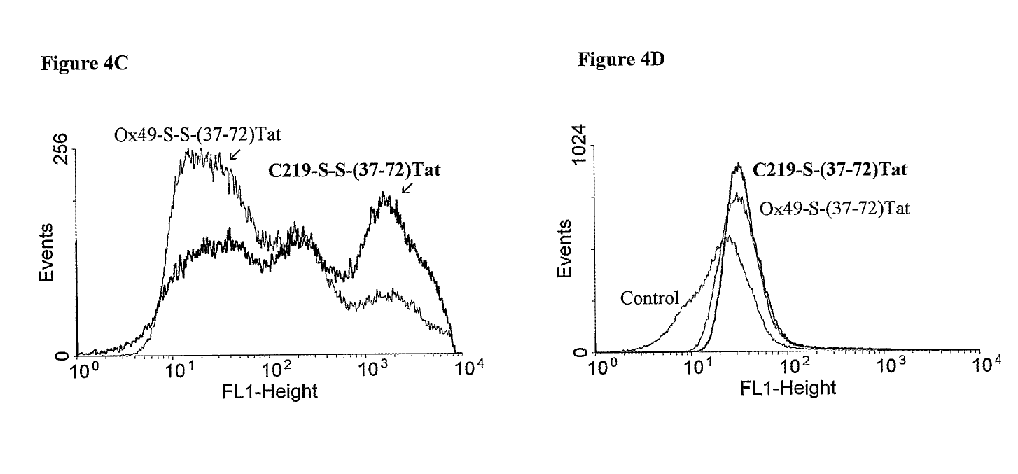

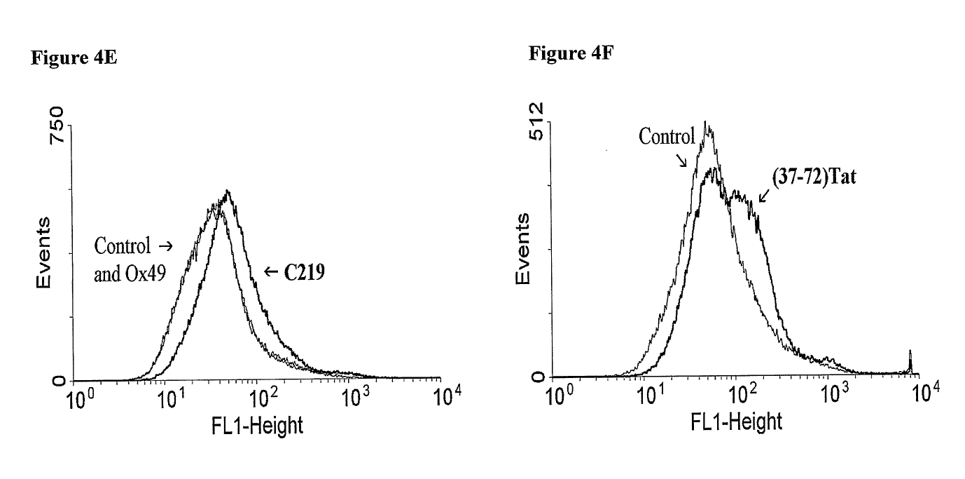

The effect of different C219-(37-72)Tat conjugates, of free, non-conjugated (37-72)Tat, and of free, non-conjugated C219, on the outward transport of esterified calcein from drug resistant CH(R)B30 cells was studied using flow cytometry as demonstrated in FIGS. 4A-4F. Incubation of CH(R)B30 cells with 0.25 μM PDPH-conjugated C219-S-S-(37-72)Tat for 130 min enhanced cellular fluorescence (intracellular calcein accumulation), shifting the histogram more than one order of magnitude to the right of the controls (FIG. 4A). Controls included the non-specific free Mab Ox49 and the reducible, Ox49-S-S-(37-72)Tat conjugate (FIG. 4A). Similar results were obtained in all three experiments. These findings also demonstrate that all cells in the population were affected by the anti-ABCB1-disulfide-Tat conjugate. Addition of 25 μM verapamil (a known inhibitor of P-gp) for 15 minutes to ascertain the responsiveness of CH(R)B30 cells also demonstrated enhanced calcein accumulation in the whole population. Concentration-response curves were constructed from two experiments with the specific C219-S-S-(37-72)Tat. The curve in FIG. 4B, showing concentration-action dependence of C219-S-S-(37-72)Tat after 210 min of incubation (filled triangles—medians; open triangles—geometric means of the same curves; error bars of the medians were S 3% and are not shown), shows a steep rise with a maximum between 10-30 nM of the conjugate. A difference between flow cytometry curve shifts of the specific C219-S-S-(37-72)Tat and the non-specific Ox49-S-S-(37-72)Tat conjugate was still evident after 14.5 hours of incubation, at 1 μM, as demonstrated in FIG. 4C).

The thioether conjugate C219-S-(37-72)Tat is ABCB1 specific in its Mab part, but its thioether bond cannot be reduced inside cells. After 3 h of incubation at 1 μM it produced a small shift in the flow cytometry histogram (FIG. 4D) which did not differ from the shift exerted by the nonspecific Ox49-S-S-(37-72)Tat disulfide conjugate.

Incubation with the free, specific, conjugate component C219 (0.2 μM for 125 minutes) shifted the histogram slightly to the right (FIG. 4E). The shift by the other conjugate component (37-72)Tat (1 μM for 210 min) was also small (FIG. 4F).

Incubation with the highest (1 μM) concentrations of conjugates had no effect on the number of propidium iodide positive cells (less than 5%), indicating no detrimental effect on cell viability during the course of the experiments.

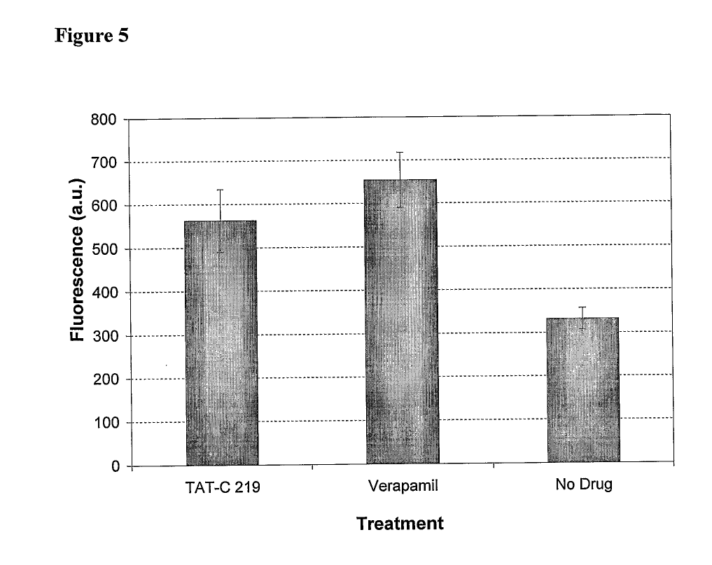

A model of T-cell lymphoma in mice (Hochman et al. 1984) was also used to test the ability of the conjugates of the present invention to inhibit MDR resistance. Two cell lines (HU-1 and HU-2, Galski et al. 1995) express the MDR transporter exogenously and are multi-drug resistant. The effect of the conjugate on one of these cell lines (HU-2) was also examined. Confirming the results obtained with CHO cells (FIG. 4), Drug-resistant HU-2 lymphoma cells were treated with Calcein-AM in the presence of conjugate or verapamil as described in materials and methods. As can be seen in FIG. 5, the presence of the conjugate significantly increased the fluorescence of the calcein over control cells to levels comparable to those found in the presence of verapamil. 1×10⁴ cells were incubated for 4 hours with 10 m of the conjugate or for 10 min with 10 mM Verapamil. 250 nM Calcein AM was then added and the fluorescence measured after 30 minutes as described in Materials and Methods. Relative florescence is shown in arbitrary units (a.u.).

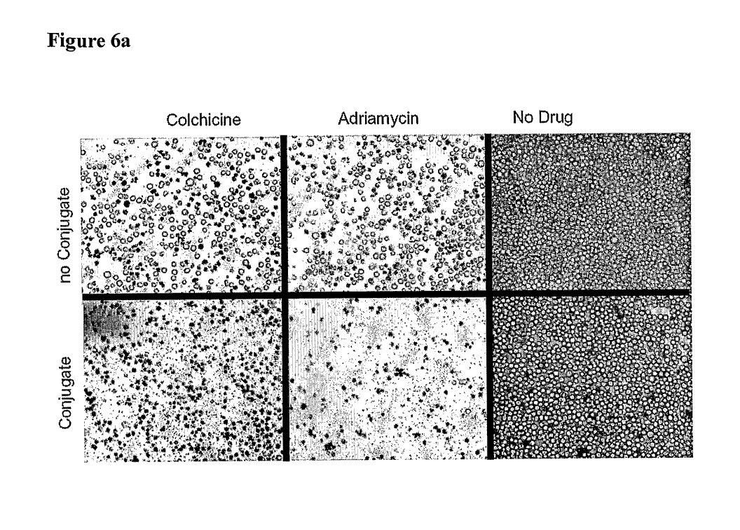

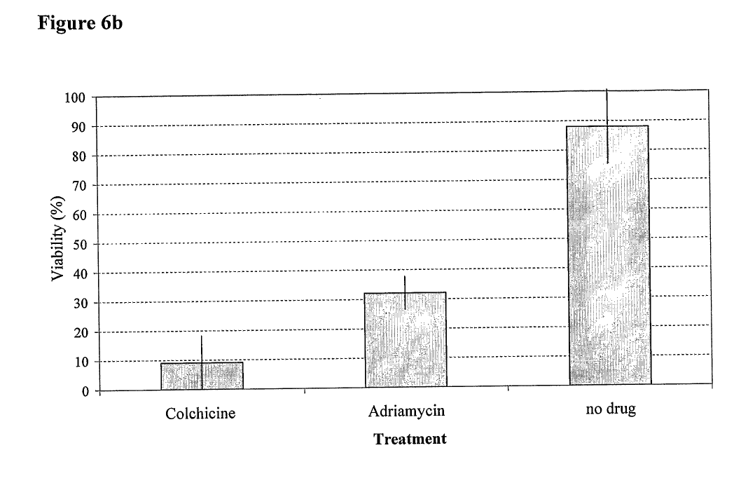

It was further determined if the conjugate could render HU-2 cells sensitive to chemotherapeutic drugs. The efficacy of the conjugate was tested using colchicine and adriamycin. As can be seen in FIG. 6A, although HU-2 cells remain viable in the presence of both colchicine and adriamycin, the addition of the conjugate renders them sensitive to both drugs. 1×10⁵ cells were incubated with 10 μg of the conjugate for 4 hours. 2 μg/ml of Adriamycin or 1 μg/ml of colchicine was then added and the cells incubated for a further 96 hrs. Light microscopy of treated and control cells is shown in FIG. 6A. These results are quantified in FIG. 6B showing viable cells from 4 independent experiments using 3 different batches of conjugate were counted. (Student's T-test of either drug vs. control, <0.008). The results were normalized to the number of viable cells in the presence of each drug without the conjugate. As can be seen, the drugs in the presence of conjugate were much more effective in inducing cell death (colchicine—91%; adriamycin—68%) than in its absence. The conjugate alone inhibits cells growth by up to 10% (˜90% viability).

### Summary of Results

It was demonstrates that the C219 anti-MDR monoclonal antibody linked through a disulfide bridge to a translocator peptide can enter colchicine-resistant cells and reduce the biological activity of membrane-bound antigens like MDR, as shown here with the C219-S-S-(37-72)Tat conjugate. The shift in the histogram of drug-resistant cells (FIG. 4) subjected to this conjugate demonstrates that essentially all cells were affected. The inhibitory effect is specific since free, non-conjugated C219, free non-conjugated (37-72)Tat, as well as the control conjugate Ox49-S-S-(37-72)Tat (non relevant mouse anti rat antibody) had only a marginal effect. Therefore, because C219 reacts in an immune-specific manner with intracellular epitopes of MDR-1 and MDR-3 while Ox49 does not, we suggest that the reduction of MDR activity in colchicine-resistant CH(R)B30 cells by C219-S-S-(37-72)Tat is largely brought about by an immune reaction between C219 and the intracellular epitopes of ABCB1 (VVQAALD and VVQEALD).

Another essential property of a C219-(37-72)Tat conjugate for reducing the MDR activity is the cleavable linkage between the components. It is shown that a conjugate containing a thioether bridge, which is a poor target for intracellular enzymes, had no action beyond that seen in the control experiments, while the disulfide bridge containing conjugate, being a good target for protein disulfide reductase, was active. This may indicate that for an unrestricted reaction with its membrane-bound epitope, the antibody must be separated from the translocating peptide (at least in the case of ABCB1) after internalization into the target cells by intracellular reduction of the linker disulfide bond.

It was also shown that the conjugate was effective in reversing the resistance to chemotherapeutic drugs in MDR-expressing cells (FIG. 6). It is noteworthy that the effect of the conjugate was obtained by a single treatment over the five day course of the experiment. The conjugate itself had little effect on the growth or on the viability of the cells.

### In Vivo Studies

Based on the findings that the ABCB1 transporter can be inhibited from inside live cells, using a specific (37-72)Tat-S-S-anti-ABCB1 immune conjugate, in vivo experiments are performed to assess if the conjugate can reverse multi-drug resistance conferred on transformed cells by this embedded ABC transporter.

Protocol for the Treatment of Mice with Drug Resistant Tumors:
Two million HU-2 cells are inoculated into Balb/C mice (intraperitoneal, in 100 microlitre) at day 8 postnatal. Mice are then divided into four groups (A-D) that receive the following treatments:
A. Inoculation of 10 micrograms of the conjugate C219-Tat in a volume of 50 microlitres plus Adriamycin (5 mg/kg body weight) in a volume of 50 microlitres (a total of 100 microlitres);
B. Inoculation of 10 micrograms of C219-Tat in a volume of 50 microlitres plus 50 microlitres PBS;
C. Inoculation of Adriamycin (5 mg/kg body weight) in a volume of 50 microlitres plus 50 microlitres PBS;
D. Inoculation of 100 microlitres PBS.
   The protocol includes five inoculations every other day and follow up of the mice.

### REFERENCES

Alahari, S. K., DeLong, R., Fisher, M. H., Dean, N. M., Viliet, P., and Juliano, R. L., 1998, Novel chemically modified oligonucleotides provide potent inhibition of P-glycoprotein expression. J Pharmacol Exp Ther 286, 419-28.
Borst P, Elferink R O., 2002, Mammalian ABC transporters in health and disease. Annu Rev Biochem 71:537-92.
Bruggemann et al. BioTechniques, 1991, 10, 202-204, 206, 208-209.
Cole et al., 1984, Mol. Cell. Biol., 62:109.
Cucco, C., and Calabretta, B., 1996, In vitro and in vivo reversal of multidrug resistance in a human leukemia-resistant cell line by mdr1 antisense oligodeoxynucleotides. Cancer Res 56, 4332-7.
Endicott J A, Sarangi F, Ling V. 1991, Complete cDNA sequences encoding the Chinese hamster P-glycoprotein gene family. DNA Seq; 2:89-101.
Essodaigui et al., 1998 Biochemistry 37 (8) 2243-50; Essodaigui et al., 1999 Mol Biochem Parasitol. 100 (1) 73-84
Fardel O, Lecureur V, Guillouzo A. The P-glycoprotein multidrug transporter, 1996, Gen. Phamacol., 27:1283-91.
Fawell S, et al. 1994, Proc. Natl. Acad. Sci. (USA) 91 664-668.
Frankel A D, Pabo C O., 1988, Cellular uptake of the tat protein from human immunodeficiency virus, Cell; 55:1189-93.
Galski et al. 1995, Eur J Cancer, 31A (3) 380-8.
Georges E, Bradley G, Gariepy J, Ling V., 1990, Detection of P-glycoprotein isoforms by gene-specific monoclonal antibodies. Proc. Nat. Acad. Sci. (USA); 87:152-56.
Georges E, Zhang J T, Ling V., 1991, Modulation of ATP and drug binding by monoclonal antibodies against P-glycoprotein. J Cell Physiol., 148:479-84.
Gottesman M M, Fojo T, Bates S E., 2002, Multidrug resistance in cancer: role of ATP-dependent transporters. Nature Rev Cancer 2:48-58.
Haus-Cohen, M., Assaraf, Y. G., Binyamin, L., Benhar, I., and Reiter, Y., 2004, Disruption of P-glycoprotein anticancer drug efflux activity by a small recombinant single-chain Fv antibody fragment targeted to an extracellular epitope, Int J Cancer 109, 750-8.
Heike, Y., Kasono, K., Kunisaki, C., Hama, S., Saijo, N., Tsuruo, T., Kuntz, D. A., Rose, D. R., and Curiel, D. T., 2001, Overcoming multi-drug resistance using an intracellular anti-MDR1 sFv. Int J Cancer 92, 115-22.
Hermanson, G. T. 1996: Bioconjugate Techniques. Academic Press, San Diego
Higgins C F., 2001, ABC transporters: physiology, structure and mechanism—an overview. Res Microbiol., 152:205-10.
Hochman et al., 1984, J. Cell Biol. 4 (pt 1) 1282-8
Hollo, Z., Homolya, L., Davis, C. W., and Sarkadi, B., 1994, Calcein accumulation as a fluorometric functional assay of the multidrug transporter. Biochim. Biophys. Acta. 1191, 384-8.
Homolya, L., Hollo, Z., Germann, U. A., Pastan, I., Gottesman, M. M., and Sarkadi, B., 1993, Fluorescent cellular indicators are extruded by the multidrug resistance protein., J. Biol. Lo Chem. 268, 21493-6.
Kabouridis P S et al., 2003, Trends in Biotechnology 21(11) 49-503.
Kartner N, Evernden-Porelle D, Bradley G, Ling V., 1985, Detection of P-glycoprotein in multidrug-resistant cell lines by monoclonal antibodies. Nature 316:820-23.
Kohler, G. and Milstein, C., 1975, Nature, 256:495-497.
Kokubu N, Cohen D, Watanabe T., 1997, Functional modulation of ATPase of P-glycoprotein by C216, a monoclonal antibody against P-glycoprotein. Biochem Biophys Res Commun; 230:398-401.
Mann D A, Frankel A D., 1991, Endocytosis and targeting of exogenous HIV-1 Tat protein. Embo. J, 10:1733-39.
Materna, V., Liedert, B., Thomale, J., and Lage, H., 2005, Protection of platinum-DNA adduct formation and reversal of cisplatin resistance by anti-MRP2 hammerhead ribozymes in human cancer cells. Int J Cancer 115, 393-402.
McBurney M W, Whitmore G F., 1974, Isolation and biochemical characterization of folate deficient mutants of Chinese hamster ovary cells, Ce11; 2:173-82.
Mie, M., Takahashi, F., Funabashi, H., Yanagida, Y., Aizawa, M., and Kobatake, E., 2003, Intracellular delivery of antibodies using TAT fusion protein A. Biochem Biophys Res Commun 310, 730-4.
Morrison et al., 1984 Proc. Natl. Acad. Sci., 81:6851.
Motomura, S., Motoji, T., Takanashi, M., Wang, Y. H., Shiozaki, H., Sugawara, I., Aikawa, E., Tomida, A., Tsuruo, T., Kanda, N., and Mizoguchi, H., 1998. Inhibition of P-glycoprotein and recovery of drug sensitivity of human acute leukemic blast cells by multidrug resistance gene (mdr1) antisense oligonucleotides. Blood 91, 3163-71.
Muller G, Laurent G, Ling V., 1995, P-glycoprotein stability is affected by serum deprivation and high cell density in multi-drug resistant cells. J Cell Physiol., 163:538-44.
Orlandi et al., 1989, Proc. Natl. Acad. Sci. 86:3833.
Osada, H., Tokunaga, T., Abe, Y., Asai, S., Miyachi, H., Hatanaka, H., Tsugu, A., Kijima, H., Yamazaki, H., Shima, K., Ueyama, Y., Osamura, Y., and Nakamura, M., 2003, Reversal of drug resistance mediated by hammerhead ribozyme against multidrug resistance-associated protein 1 in a human glioma cell line, Int J Oncol 22, 823-7.
Paterson, D. J., Jefferies, W. A., Green, J. R., Brandon, M. R., Corthesy, P., Puldavec, M., and Williams, A. F., 1987, Antigens of activated rat T lymphocytes including a molecule of 50,000 Mr detected only on CD4 positive T blasts. Mol Immunol 24, 1281-90.
Potocky, T. B., Menon, A. K., and Gellman, S. H., 2003, Cytoplasmic and nuclear delivery of a TAT-derived peptide and a beta-peptide after endocytic uptake into HeLa cells. J Biol Chem 278, 50188-94.
Rasband, W. S. Image J, 1997-2004, National Institutes of Health, Bethesda, Md., USA, http://rsb.info.nih.gov/ij/.
Ratner L, Haseltine W, Patarca R, Livak K J, Starcich B, Josephs S F et al., 1985, Complete nucleotide sequence of the AIDS virus, HTLV-III. Nature, 313:277-84.
Robert, J., and Jarry, C., 2003, Multidrug resistance reversal agents, J. Med. Chem. 46, 4805-17.
Scanlon, K. J., Ishida, H., and Kashani-Sabet, M., 1994, Ribozyme-mediated reversal of the multidrug-resistant phenotype. Proc Natl Acad Sci USA 91, 11123-7.
Sodroski, J. et al., 1986, Nature 321, 197-209.
Stein, W. D., 2002, Reversers of the multidrug resistance transporter P-glycoprotein. Curr Opin Investig Drugs 3, 812-7.
Stein S, Weiss A, Adermann L, Lazarovici P, Hochman J, Wellhöner H., 1999, A disulfide conjugate between anti-tetanus antibodies and HIV (37-72)Tat neutralizes tetanus toxin inside chromaffin cells, FEBS LETTERS, 456:383-86.
Suzuki et al., 2002, J. Biol. Chem. 277, 2437-43.
Tarasova, N. I., Seth, R., Tarasov, S. G., Kosakowska-Cholody, T., Hrycyna, C. A., Gottesman, M. M., and Michejda, C. J., 2005, Transmembrane inhibitors of P-glycoprotein, an ABC transporter. J Med Chem 48, 3768-75.
Terwillinger, E. et al., 1988, J. Virol 62, 655-658.
Tsuruo T, Iida H, Tsukagoshi S, Sakurai Y., 1981, Overcoming of vincristine resistance in P338 leukemia in vivo and an vitro through enhanced cytotoxicity of vincristine and vinblastine by verapamil. Cancer Res. 41:1967-72.
Ueda K, et al., 1987, J. Biol. Chem. 262:505-508.
Varadi A, Szakas G, Bakos E, Sarkadi B., 2002, P glycoprotein and the mechanism of multidrug resistance. Novartis Found Symp. 243:54-65.
Young, I. T., 1977, Proof without prejudice: use of the Kolmogorov-Smirnov test for the analysis of histograms from flow systems and other sources, J Histochem Cytochem, 25, 935-41.

1.-34. (canceled)

35. A conjugate comprising: (i) an antibody, or a fragment thereof comprising at least the antigen-binding portion, capable of binding an intracellular epitope of an MDR protein within intact cells, and inhibiting said protein activity, (ii) a cell entering moiety; and optionally (iii) a linker connecting (i) and (ii).

36. The conjugate according to claim 35, wherein the MDR protein is an ATP-binding cassette (ABC) transporter selected from the group consisting of: MDR1 (ABCB1, P-gp), MRP4 (ABCC4), MRP5 (ABCC5), MRP1 (ABCC1), MRP2 (ABCC2), MRP3 (ABCC3), and MXR/BCRP/ABC-p (ABCG2).

37. The conjugate according to claim 35, wherein the ABC transporter is MDR1 (ABCB1, P-gp).

38. The conjugate according to claim 37, wherein the antibody or antibody fragment is directed against a MDR-1 epitope comprising a sequence selected from VQAALD (SEQ ID NO:1) and VQEALD (SEQ ID NO:2).

39. The conjugate according to claim 38, wherein the antibody is the monoclonal antibody C219.

40. The conjugate according to claim 39, wherein (i) is linked to (ii) via a carbohydrate moiety of (i).

41. The conjugate according to claim 37, wherein the intracellular epitope of the MDR protein is within the (596-636) MDR1 fragment having the sequence: VRNADVIAGFDDGVIVEKGNHDELMKEKGIYFKLVTMQTAGNEVE (SEQ ID NO:3).

42. The conjugate according to claim 35, wherein the antibody is a monoclonal antibody.

43. The conjugate according to claim 35, wherein the cell entering moiety is the cationic protein transduction domain (PTD) HIV-1 Tat.

44. The conjugate according to claim 43, wherein the HIV-1 Tat is the HIV-1(37-72) Tat fragment (SEQ ID NO:4).

45. The conjugate according to claim 35, wherein the linker comprises a cleavable sequence cleaved by intracellular enzymes over-expressed in cancer cells.

46. The conjugate according to claim 35, wherein the linker comprises a protease specific cleavable sequence, which is more abundant in malignant cells or secreted by malignant cells more than normal cells.

47. The conjugate according to claim 35, wherein (i) and (ii) are connected via a disulfide bond.

48. The conjugate according to claim 35, comprising (i) a monoclonal antibody against an intracellular epitope of MDR1 (ABCB1, P-gp), or a fragment thereof comprising at least the antigen-binding portion; (ii) an HIV-1(37-72) Tat fragment of SEQ ID NO:4; and optionally (iii) a linker connecting (i) and (ii).

49. A pharmaceutical composition comprising a pharmaceutically acceptable carrier and as an active ingredient a conjugate according to claim 35.

50. The pharmaceutical composition according to claim 49, further comprising an anti-cancer agent of the type that is expelled by the specific MDR (ABC transporter) against which the antibody part of the conjugate is directed.

51. A kit comprising the pharmaceutical composition of claim 49 and in a separate vessel at least one anti-cancer agent or at least one medicinal agent to be transported through the blood brain barrier.

52. A method of inhibiting MDR activity in MDR cells; the method comprises providing the MDR cells with an amount of a conjugate according to claim 35, the amount being sufficient to inhibit MDR activity in the cells.

53. A method for circumventing or treating MDR cancer, the method comprises providing a subject in need an amount of the conjugate of claim 35, the amount being effective to inhibit MDR activity in cancer cells in said subject.

54. The method according to claim 53, wherein the cancer is selected from colon, kidney, adrenocortical and hepatocellular cancers; breast cancer, acute myelogenous leukemia (AML), chronic lymphocitic leukemia (CLL), pro-lymphocitic leukemia, oesophagal carcinoma, non-small-cell lung cancers, soft-tissue sarcomas and osteosarcomas.

55. A method for sensitizing an MDR cancer to anti-cancer drugs, the method comprises administering said subject with a therapeutically effective amount of the conjugate of claim 35 in combination with said anti-cancer therapy, the amount being effective to sensitize the MDR cancer cells to one or more drugs forming part of the anti-cancer therapy.

56. A method of preventing the development of MDR in a subject undergoing anti-cancer therapy, comprising administering to the subject prior or at the time of the anti-cancer therapy, a therapeutically effective amount of the conjugate of claim 35.

57. A method for enhancing the transport of a medicinal agent through the blood brain barrier the method comprising administering to a subject in need of such treatment with an amount of a conjugate of claim 35, the amount being sufficient to enhance the transport of the medicinal agent through the blood brain barrier.

58. The method of claim 57 comprising inhibiting P-gp activity in the blood brain barrier.
